SEQUENCE LISTING (12 sequences)

SEQ ID NO: 1 - DNA, 20 bp, Artificial Sequence
  Made in a lab (primers)
agctggggagaaaactgggg

SEQ ID NO: 2 - DNA, 30 bp, Artificial Sequence
  Made in a lab (primers)
tgaagcacaaacaaatggggaaactgaaca

SEQ ID NO: 3 - DNA, 44 bp, Artificial Sequence
  Made in a lab (primers)
tgaagcacaaacaaatggggaaactgaacaggctggctggagtc

SEQ ID NO: 4 - DNA, 20 bp, Artificial Sequence
  Made in a lab (primers)
tgcccagaagacagatacat

SEQ ID NO: 5 - DNA, 19 bp, Artificial Sequence
  Made in a lab (primers)
gcttcatcataggcacctt

SEQ ID NO: 6 - DNA, 34 bp, Artificial Sequence
  Made in a lab (primers)
gcttcatcataggcaccttggtggtcagctggag

SEQ ID NO: 7 - DNA, 19 bp, Artificial Sequence
  Made in a lab (primers)
cgtggcccttcatggtgtc

SEQ ID NO: 8 - DNA, 20 bp, Artificial Sequence
  Made in a lab (primers)
gcaaaggcatcggggtagag

SEQ ID NO: 9 - DNA, 33 bp, Artificial Sequence
  Made in a lab (primers)
gcaaaggcatcggggtagagcggcgtccctgag

SEQ ID NO: 10 - DNA, 24 bp, Artificial Sequence
  Made in a lab (primers)
ttccacaggtcccacaaccgcagc

SEQ ID NO: 11 - DNA, 15 bp, Artificial Sequence
  Made in a lab (primers)
cgtccagcaatgagt

SEQ ID NO: 12 - DNA, 29 bp, Artificial Sequence
  Made in a lab (primers)
cgtccagcaatgagtggctgttccaggga

(19) United States
(12) Patent Application Publication
(10) Pub. No.: US 2005/0244448 A1
(43) Pub. Date: Nov. 3, 2005

(54) Diagnosis and treatment of disorders of collagen and elastin metabolism

(75) Inventors: Bertha Chen, Menlo Park, CA (US); Yan Wen, San Jose, CA (US); Mary Lake Polan, Palo Alto, CA (US)

Correspondence Address: SPECKMAN LAW GROUP PLLC, 1501 WESTERN AVE, SEATTLE, WA, 98101, US

(73) Assignee: The Board of Trustees of the Leland Stanford Jr. University, Palo Alto, CA (US)

(21) Appl. No.: 11/082,460

(22) Filed: Mar. 16, 2005

Related U.S. Application Data
(63) Continuation-in-part of application No. 10/684,539, filed on Oct. 14, 2003.
(60) Provisional application No. 60/554,170, filed on Mar. 16, 2004.
(60) Provisional application No. 60/419,007, filed on Oct. 14, 2002.

(51) Int. Cl. A61F 13/00

(52) U.S. Cl. 424/422

(57) ABSTRACT

This application discloses methods for diagnosing and treating SUI. The treatment methods are directed to inhibiting elastolysis in affected tissues and correcting imbalances in the expression of elastase enzymes and their inhibitors.

### CROSS REFERENCE TO RELATED APPLICATIONS

This application claims the benefit of priority under 35 U.S.C. §119(e) to U.S. Provisional Application No. 60/554,170, filed Mar. 16, 2004, which is incorporated by reference herein in its entirety. This application is a continuation-in-part of U.S. application Ser. No. 10/684,539, filed Oct. 14, 2003, which is incorporated by reference herein in its entirety, and which claims the benefit of priority to U.S. Provisional Application No. 60/419,007, filed Oct. 14, 2002.

### STATEMENT OF GOVERNMENT FUNDING

This work was supported by a grant from the National Institutes of Health (RO1AG017907). The Government may have certain rights in this invention.

### FIELD OF THE INVENTION

This invention relates to stress urinary incontinence and other disorders of collagen and elastin metabolism characterized by imbalances in the levels of expression and activities of tissue elastases and elastase inhibitors. More particularly, this invention relates to the diagnosis and therapy (including prophylaxis) of stress urinary incontinence.

### BACKGROUND

Urinary incontinence and pelvic floor dysfunction can become major health and quality-of-life issues as women age through the reproductive and menopausal years. Currently, American women can expect an 11.1% lifetime risk of surgery for pelvic floor dysfunction by age 80 and of those who do undergo surgery, nearly 30% will require a repeat operation for recurrence (Olsen et al, 1997). Known risk factors for the development of SUI include higher parity, prior pelvic trauma, high body mass index (BMI), and race (Sampselle et al, 2002; Howard et al, 2000). While the mode of delivery affects the prevalence of urinary incontinence, pregnancy itself may cause this condition because of mechanical and/or hormonal changes (Rortveit et al, 2003). However, despite numerous epidemiologic studies on urinary incontinence and prolapse, the underlying pathophysiology is poorly understood.

Structurally, the female lower urinary system is supported by pelvic muscles, ligaments, and the bony pelvis, all of which are constantly subjected to stresses and trauma including childbirth. Collagen in the extracellular matrix of pelvic supporting tissues provides strength and mechanical stability, while elastin provides supporting tissues with the ability to recoil after physical stress and distension.

The extracellular matrix is a complex mixture of long-chain proteins, such as collagen, elastin, laminin, and gelatin, each with specific structural properties. Elastin is primarily laid down during fetal development and is rarely synthesized in adult tissues (Prosser et al, 1988). If damaged or destroyed, metabolically repaired elastin may be malformed and dysfunctional (Finlay et al, 1996).

Changes in collagen and elastin metabolism, brought about by environmental and genetic factors, are associated with disorders and conditions such as emphysema, vascular aneurysm, and age-related changes in skin. Changes in gene expression in pelvic supporting tissues of women during pregnancy and menopause, and in women with SUI/POP, strongly suggest that extracellular matrix degradation is an essential etiological factor in female pelvic floor dysfunction (see, e.g., Yamamoto et al, 1997; Chen et al, 2002; Chen et al, 2003; Visco et al, 2003; Chen et al, 2004).

Elastin-degrading enzymes (elastases) include the serine proteases (e.g., members of the neutrophil elastase family), cysteine proteases (e.g., cathepsins L, S, and K) and matrix metalloproteinases (MMPs). MMP-2, MMP-9 and MMP-12 are the MMPs primarily responsible for elastin breakdown, but are less potent elastolytic enzymes than either the serine or cysteine proteases (Werb et al, 1982). Endogenous elastase inhibitors that are active against serine proteases include elafin, secretory leukocyte protease inhibitor (SLIP), alpha 2-macroglobulin and alpha-1-antitrypsin (ATT). Tissue inhibitors of MMPs include, for example, TIMP-1 and TIMP-2.

The studies disclosed in this application suggest that reproductive steroid and peptide hormones, cytokines and growth factors modulate elastolytic changes in pelvic support tissues.

Diagnostic tests for SUI, based on changes in expression ratios of matrix metalloproteinase enzymes (MMPs) and their tissue inhibitors (TIMPs) are disclosed and claimed in U.S. Pat. No. 6,420,119. Methods of diagnosis and treatment of SUI, based on unique gene expression patterns in women with SUI, are disclosed and claimed in U.S. patent application Ser. No. 10,684,539.

The present application discloses methods for diagnosis of SUI based on changes in elastase and anti-elastase activities in pelvic support tissues of women with SUI, methods of treating SUI and other disorders of elastin and collagen metabolism, and assays for modulators of elastolytic activity.

### BRIEF DESCRIPTION OF THE FIGURES

FIG. 1. Total elastin (A) and collagen (B) in secretory phase vaginal cuff tissue of control and incontinent women.

FIG. 2. Periurethral vaginal tissue extracts from continent or from age- and phase-matched SUI patients analyzed by Western Blots for TIMP-1 expression.

FIG. 3. Western blot of TIMP-1 from control (A) and SUI (B) pelvic fibroblasts as a function of increasing concentrations of 17-β estradiol.

FIG. 4. Mean elastolytic activity in SUI/POP group (N=6, mean ±SE, 0.016±0.004) compared to mean elastolytic activity in control group (N=6, mean ±SE, 0.013±0.003).

FIG. 5. QC-PCR analysis of human neutrophil elastase mRNA from vaginal wall tissue in SUI/POP (mean±SE, 52.12±23.92) and control patients (mean ±SE, 109.51±50.38). Lane 1, DNA ladder; lane 2-11 (top), PCR products from SUI patients; lane 2-11 (bottom), PCR products from control patients. The ratios of the densities of target cDNA band (580bp) to competitive cDNA band (333 bp) were compared with the values obtained from the standard curve.

FIG. 6. QC-PCR analysis of cathepsin K mRNA from vaginal wall in both SUI/POP (mean ±SE, 2.79±1.6) and control patients (mean ±SE, 2.65±1.1). Lane 1, DNA ladder; lane 2-9, PCR products from SUI patients; lane 10-16, PCR products from control patients.

FIG. 7. Cathepsin K Western blot analysis on vaginal wall tissue. Lane 1-3 (S1-S3), protein from SUI/POP patients; lane 4-6 (C1-C3), protein from control patients.

FIG. 8. (A) Western blot analysis of alpha-1 antitrypsin in vaginal wall tissue. Lane 1-5 (S1-S5), protein from SUI/POP patients; lane 6-10 (C1-C5), from control patients. The same blot was reprobed with mouse anti-human TGFβ3 monoclonal antibody to confirm that equal amounts of protein were loaded in each lane. Plasma alpha-1 antitrypsin was used as positive control. (B) Protein levels of alpha-1 antitrypsin in SUI/POP patients (mean±SE, 0.42±0.078) were significantly lower (p<0.003) than those in the control group (mean ±SE, 1.58±0.27).

FIG. 9. (A) QC-PCR analysis of alpha-1 antitrypsin mRNA from vaginal wall in both SUI/POP and control patients. Lane 1-11 (top), PCR products from SUI patients; lane 1-9 (bottom), PCR products from control patients. (B) Alpha-1 antitrypsin mRNA level in SUI patients (N=11, mean±SE, 26.6±7.5) compared to control patients (N=9, mean±SE, 117.2±37.4). Eight out of nine case-control pairs showed lower alpha-1 antitrypsin mRNA levels in cases compared to controls.

FIG. 10. Quantitative competitive RT-PCR analysis of TGF-β3 mRNA in vaginal tissue from 13 incontinent and 11 continent follicular phase, age, BMI and parity matched women without significant prolapse. The ratio of SUI/control TGF-β3 expression is 1.6.

FIG. 11. Cultured proliferative phase control human vaginal fibroblasts exposed to physiologic levels of relaxin secrete significantly increased amounts of MMP-9 and MMP-2.

FIG. 12. Cultured human vaginal fibroblasts exposed to increasing levels of human relaxin show significant decreases in TIMP-1 niRNA expression, as determined by quantitative RT-PCR. Each bar represents the mean±SD of 4 individual experiments.

FIG. 13. Secretory phase vaginal cuff fibroblasts from a continent subject cultured for 48 hours with relaxin (0-100 ng/ml). Both cells (A) and conditioned media (B) show significant increases in total elastase activity.

FIG. 14. Western blot analysis of cultured vaginal fibroblasts shows secretion of α1-antitrypsin into conditioned media. Relaxin stimulates expression of α-1-antitrypsin in control cells (A) but inhibits expression in cells from a matched SUI patient (B).

FIG. 15. Percent inhibition of total elastase activity in relaxin-stimulated, cultured vaginal fibroblasts by ATT. Secretory phase vaginal fibroblasts from a stress incontinent woman were cultured for 48 hours with relaxin (100 ng/ml), then lysed and incubated for 1 hour with increasing concentrations of α-1-antitrypsin.

### SUMMARY OF THE INVENTION

In one of its aspects, the present invention provides a method of treatment of SUI in women at risk for developing SUI, and in women with symptoms of SUI. The method comprises administering an effective amount of an inhibitor of elastase activity to a pelvic supporting tissue of a patient in need of treatment.

In one of its embodiments, the inhibitor is an endogenous inhibitor (e.g., alpha-1 antitrypsin(ATT), elafin, secretory leukocyte protease inhibitor(SLPI), and alpha 2-macroglobulin, TIMPs). Preferably, the inhibitor is a recombinant DNA sequence encoding an active form of the inhibitor or a functionally active fragment of the inhibitor, and is delivered to the tissue in an expressable genetic construct comprising a promoter operably linked to the DNA sequence and a termination sequence.

In another embodiment, the inhibitor is a synthetic inhibitor, preferably a small organic molecule that is formulated for intravaginal or intrauterine delivery, preferably as a slow-release formulation.

The invention encompasses treatment methods where multiple inhibitors are co-administered to the patient.

In another aspect, the invention provides a method of treatment of SUI comprising administering an effective amount of a relaxin antagonist to a pelvic supporting tissue of a patient in need of treatment. The antagonist can be a known antagonist of a relaxin receptor (e.g., a peptide, antibody or small synthetic molecule) or an inhibitor of relaxin signaling. Methods are disclosed for identifying a relaxin antagonist in a cell-based assay.

In yet another aspect, the invention provides a method for diagnosing SUI comprising incubating cultured pelvic fibroblasts from a test subject in the presence and absence of relaxin.

In one embodiment, the level of ATT protein and total elastase activity are compared in relaxin-treated and untreated cells.

In another embodiment, the method additionally comprises comparing one or more of MMP-2, MMP-9 and TIMP-2 protein expression in the presence and absence of relaxin in test cells as compared with normal cells.

In yet another embodiment, the method additionally comprises measuring MRNA levels of TIMP-1.

The treatment methods disclosed herein are intended for use in humans and non-human animals. These methods are expected to be useful for prophylactic therapy in subjects at risk for developing SUI and pelvic organ prolapse, for primary treatment of early-stage SUI, and as an adjunct to surgery in chronic advanced SUI. The methods of treatment may also be used together with the diagnostic methods disclosed in the application, or with other diagnostic methods that are known in the art.

### DETAILED DESCRIPTION OF PREFERRED EMBODIMENTS

1. Proteolysis of Collagen and Elastin in Urinary Dysfunction

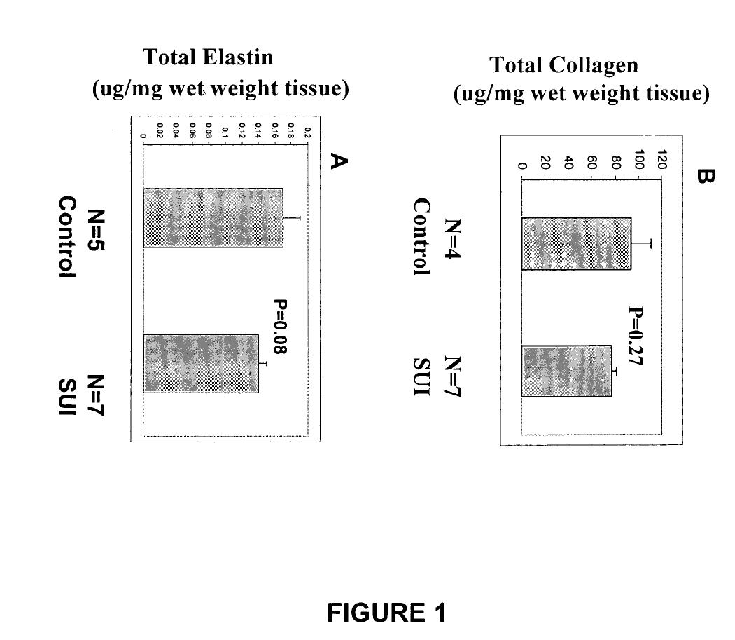

Increased collagen and elastin degradation occurs with, and may be etiologic for, stress urinary incontinence. The finding that total collagen and elastin content of vaginal tissue from continent women is greater than in tissue from patients with SUI is consistent with this hypothesis (FIG. 1). Pyridinoline crosslinks occur in both type I and type II collagen found in most connective tissues, and are believed to be important for tissue integrity. However, the evidence for changes in crosslinking in tissue with pelvic dysfunction is inconclusive and contradictory. In examining the amount of crosslinking in pelvic tissues of continent and incontinent women, we found no difference in the level of pyridinoline crosslinks (Chen et al., 2002). Incontinence and pelvic floor dysfunction may be primarily associated with a higher turnover of less mature collagen fibrils (Jackson S R et al.1996). Laminin, an extracellular matrix protein, regulates the assembly of collagens into high-order fibrils in connective tissues and has been identified as a candidate gene in the pathogenesis of certain connective tissue disorders. We have identified laminin as one of the genes whose expression is upregulated in pelvic tissues of women with SUI, compared to matched tissues from continent women (see below).

Changes in the expression of MMPs, TIMPs, and tissue elastase inhibitors, and increases in total tissue elastase activity promote proteolytic degradation of collagen and elastin of pelvic viscera. As discussed further below, these changes in expression are susceptible to modulation by hormones and growth factors.

A. TIMP Expression in Pelvic Tissue

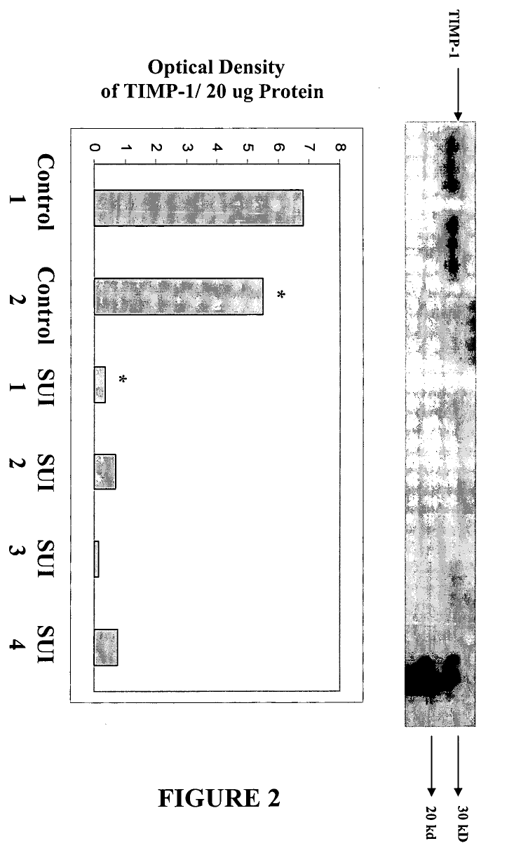

TIMP-1 is a stoichiometric inhibitor of MMP-1 and MMP-9. TIMP-1 protein expression is significantly reduced in periurethral vaginal tissue from stress incontinent compared to continent premenopausal women, which is consistent with previously observed decreases mRNA expression (Chen et al., 2002) (FIG. 2). The difference in means between the control and SUI groups is approximately 4, with a standard deviation of approximately 0.5. Protein expression of MMP-1 was similar in tissue from incontinent and continent women in this carefully-matched subject population.

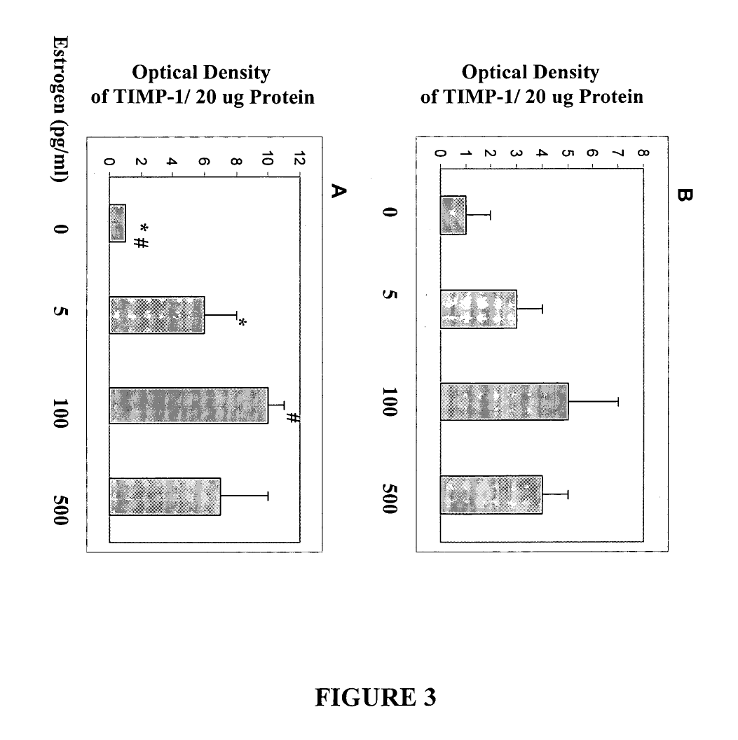

TIMP-1 protein expression was differentially modulated by 17β-estradiol in fibroblasts cultured from pelvic tissues of women with SUI and normal continent women. As shown in FIG. 3, 17β-estradiol significantly increased TIMP-1 expression in control but not SUI, fibroblasts. MMP-1 protein expression in fibroblasts cultured from both control and incontinent women was unaffected by estrogen concentration. This suggests that estrogen, at physiologic levels, may preferentially inhibit collagen degradation in continent women by increasing TIMP-1.

B. Elastolytic Activity

Elastin modulates the mechanical characteristics of supportive tissues, and decreased elastin production has been documented in fibroblasts from women with pelvic organ prolapse. The role of elastin degradation in pelvic dysfunction has received less attention, in part because of the scant availability of tissue for analysis. Not only are these pelvic areas difficult to sample, but removal of large amounts of healthy supportive tissues may result in incontinence or pelvic floor dysfunction in the asymptomatic control women. The results reported in this application were obtained using full-thickness vaginal wall tissue, which contains collagen that is similar to collagen of endopelvic fascia collagen (Keane et al, 1992, Neurourol Urodyn 11: 308-9) and elastin (Farrell et al, 2001, Obstetrics & Gynecology 98: 794-498), and is similar in gene expression and other respects to other pelvic supporting tissues.

We investigated the possibility that abnormal elastin metabolism contributes to abnormal connective tissue seen in women with pelvic floor dysfunction by assessing total elastase activity, human neutrophil elastase and cathepsin K activity, and levels of an elastase inhibitor (alpha-1 antitrypsin) in vaginal wall tissues obtained from premenopausal women with pelvic floor dysfunction and in asymptomatic controls. All participants were in the proliferative phase of the menstrual cycle.

Vaginal wall tissue samples were obtained from 12 premenopausal women with stress urinary incontinence/pelvic organ prolapse (SUI/POP) and 15 premenopausal control women without pelvic floor disorders. All SUJIPOP participants had pelvic organ prolapse equal to or greater than stage II, while all controls were less than or equal to stage I. The two groups were matched for age and body mass index (BMI), but differed with respect to parity, with higher parity in the SUI/POP group.

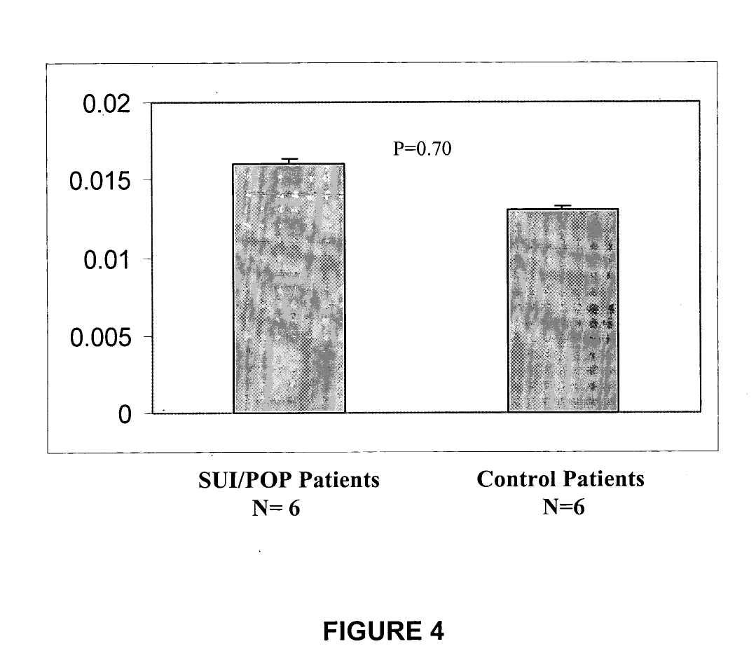

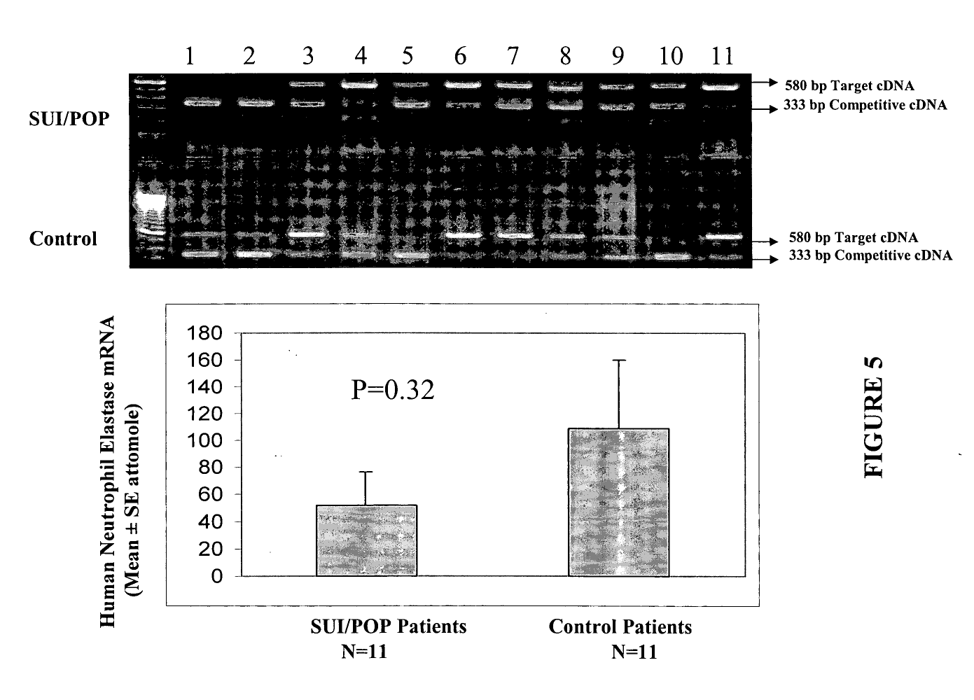

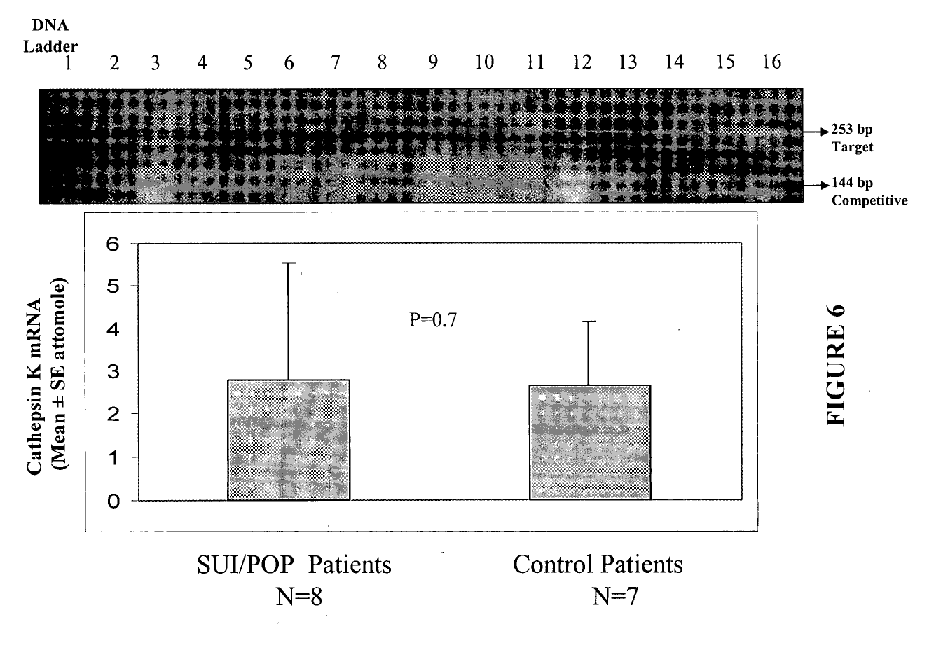

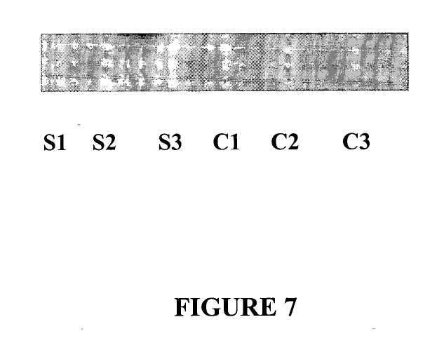

The mean elastolytic activity in the SUI/POP group was not significantly different from that in the control group (FIG. 4). There was no significant difference in expression levels of human neutrophil elastase or cathepsin K in the two groups of women, as determined by QC-PCR and Western blot analysis (FIGS. 5-7).

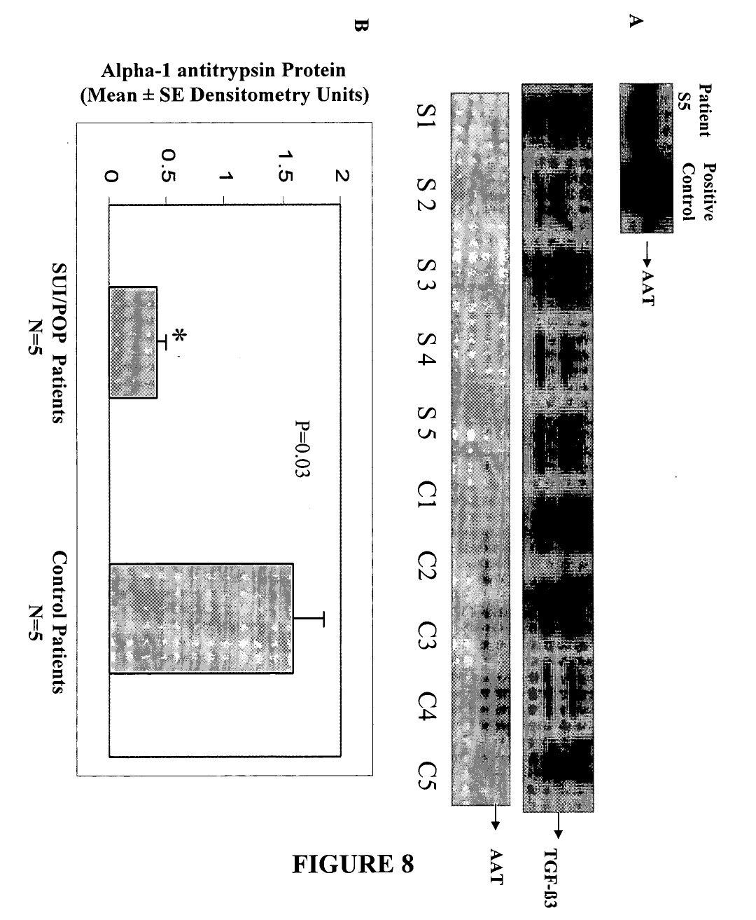

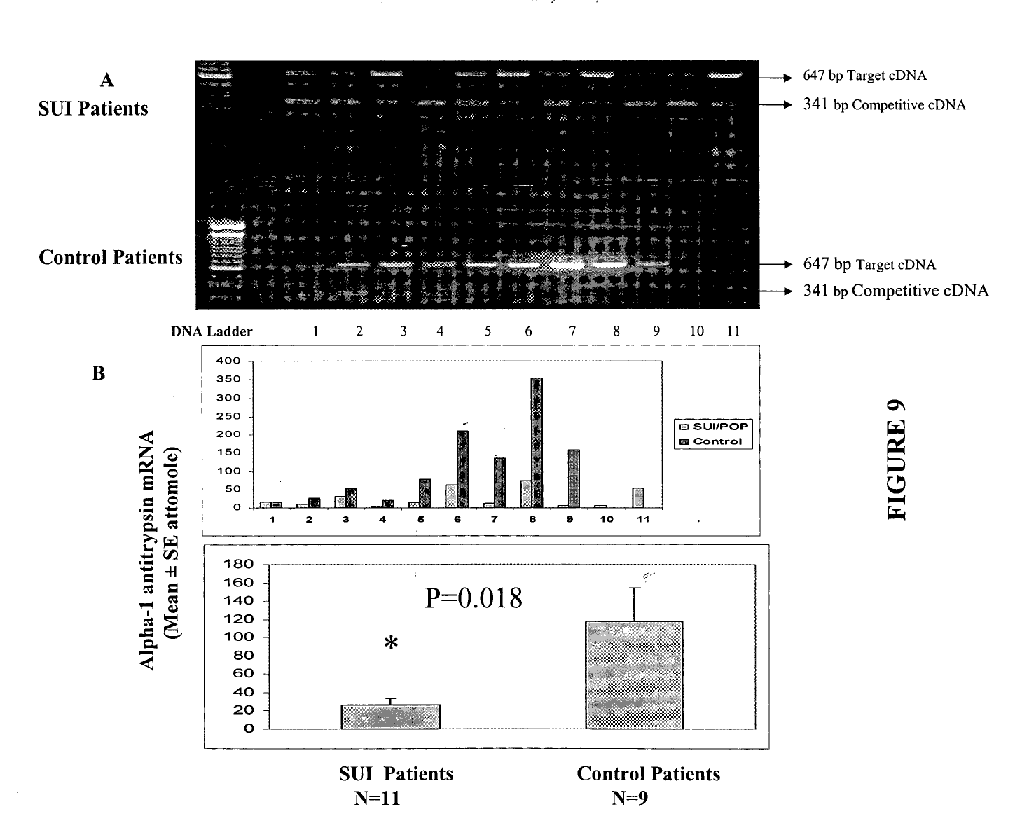

The inhibitory component of elastin turnover, however, was significantly different in vaginal wall tissues of women with SUI/POP compared to controls. Both the Western blot analysis (FIG. 8) and QC-PCR analysis (FIG. 9) of tissues from affected women showed significantly decreased levels of alpha-1 antitrypsin compared to controls (p<0.03 and p<0.018 respectively). Eight out of nine case-control pairs showed a decrease in alpha-1 antitrypsin mRNA expression in affected versus control women.

Additional studies were carried out to investigate the role of neutrophil elastase (NE) in elastin metabolism and its relative contribution to elastin degradation compared to MMP-2 in vaginal wall tissues from women with SUI. Both proliferative- and secretory phase tissues were analyzed in these studies.

Periurethral vaginal wall tissues were obtained from 22 premenopausal SUI and 29 asymptomatic control women undergoing benign gynecologic surgery. Total elastase activity was measured by generation of free amino groups from succinylated elastin. Neutrophil elastase activity was measured by using N-methoxysuccinyl-ala-ala pro-val p-nitroanailine as substrate. RT-PCR and Western blot were used respectively to evaluate neutrophil elastase (NE) and alpha-1 antitrypsin (ATT) mRNA and protein expressions. Total elastin and collagen content in vaginal wall tissues was also measured as described by Reddy et al., 1996 and Maum et al, 1973. The relative contributions of NE and MMP-2 to total elastase activity in cultured vaginal wall fibroblasts was assessed by treatment of cell extracts and culture supernatants from relaxin-stimulated secretory phase control cells with anti-NE and anti-MMP-2 antibodies.

Total elastase activity was significantly increased in secretory phase- as compared to proliferative phase tissues (p=0.007). No difference was observed between control and SUI tissues in either phase. NE mRNA and protein expressions were similar in secretory-control and SUI tissues. However, NE activity was significantly increased in the secretory compared to proliferative tissues (p=0.0001). NE activity in secretory phase SUI tissues was higher than in control tissues (p=0.022). mRNA expression of alpha-1 antitrypsin (ATT) was higher in proliferative phase controls than in secretory phase controls, whereas no difference was observed in the SUI tissues in either phase of the cycle. Protein expression of ATT in the active form was decreased in secretory-SUI tissues compared to controls (p=0.019). Total elastin and collagen contents were similar in vaginal wall tissues and were not phase-dependent. Anti-NE antibody inhibited total elastase activity by 60-70% while the inhibition by anti-MMP-2 antibody was not greater than 20%.

These results suggest that an increase in NE activity, rather than MRNA or protein expression, may contribute importantly to aberrant elastin metabolism in SUI vaginal wall and other pelvic support tissues.

Decreased expression of ATT may be important in elevated elastolysis in SUI pelvic support tissues. ATT is the major serine proteinase inhibitor in the circulation. ATT is known to inhibit elastase activity in the lung, but to our knowledge, the role of ATT in elastin metabolism in pelvic tissues has not been previously investigated. Our studies show that levels of ATT mRNA expression and active ATT protein are significantly reduced in SUI vaginal wall tissues.

C. Differential Gene Expression Patterns in SUI

Experiments were performed with oligonucleotide microarrays to analyze mRNA expression. levels in periurethral vaginal tissue biopsies from age- and menstrual cycle phase-matched normal continent women and SUI women (Chen et al 2003; and U.S. Pat. application Ser. No. 10/684,539, which is herein incorporated by reference in its entirety). Differences in patterns of gene expression were identified during the proliferative (estrogen) phase of the menstrual cycle and during the secretory (estrogen+progesterone) phase. These differences were sufficient to segregate SUI from control samples.

Proliferative Phase

Ninety candidate genes were identified in the proliferative phase as differentially expressed with a p value of <0.05. Sixty-two of the genes were up-regulated and 28 were down-regulated. The average fold increase in the up-regulated genes from SUI women compared to controls ranged from 1.3 to 4.8, while that of the down-regulated genes ranged from 1.2 to 3.1-fold.

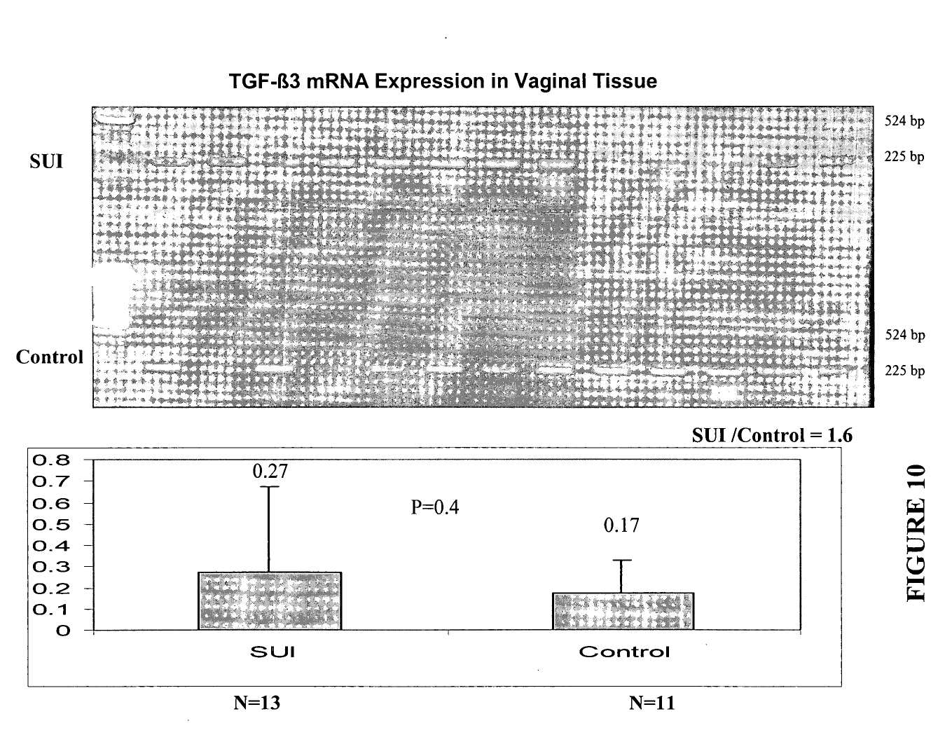

Upregulated genes included TGF-beta3, extracellular matrix molecules (e.g., laminin and collagen type VI), and myocyte function-related proteins (e.g. LIM protein and dystrophin). The expression of TGF-β3 was increased 1.7-fold in SUI tissue relative to control tissue. This increase in TGF-β3 expression is confirmed by QC-PCR measurements which showed a TGF-β3 mRNA expression ratio of 1.6 in vaginal tissue from SUI/control women (FIG. 10). TGF-β3 is a cytokine involved in cell growth regulation and differentiation, stimulation of extracellular matrix, and modulation of immune responses. The production of collagen type I and type III mRNA levels is reportedly increased by this cytokine (Lee and Nowak, 2001). These collagens are the principal components providing tensile strength to ligamentous tissues. TGF-β3 also increases the expression of pro-MMP-2 and -9 in cultured pelvic fibroblasts (Moalli et al, 2002).

Down-regulated genes that may participate in collagen metabolism and are potentially involved in increased degradation of extracellular matrix include laminin-related protein (LamA3), BPI80 (collagen XVII), serine/threonine protein kinase, type II interleukin-1 receptor, and PDGF-associated protein.

Differential expression of specific MMP and TIMP genes was demonstrated by QC-RT-PCR (Chen et al., 2002).

Clustering analysis was applied to assess the ability of the ninety up- and down-regulated candidate genes in SUI to discriminate between normal and affected individuals. In two-dimensional analyses, gene expression patterns that are similar group together, i.e., cluster. Consequently, one expects that tissues with markedly different expression patterns would form distinct clusters when sorted by expression pattern in all five tissue pairs. The clustering results support the importance of these genes for distinguishing between SUI and control tissue biological activity, and may reflect complex genetic variations that are predictive of future development of incontinence.

Secretory Phase

Fifty-eight upregulated genes and thirty-six downregulated genes were identified in SUI samples, compared with control samples, with fold-changes in expression ranging from 1.2 to 78.8. The up-regulated genes that appear to function in ECM metabolism include skin-derived protease inhibitor 3 (elafin), IL-1 receptor antagonist (IL-1RA), keratin 6, keratin 14, keratin 16 and psoriasin 1.

Elafin, a locally produced serine protease inhibitor, is an epithelial host-defense protein that is absent from normal skin but highly expressed in keratinocytes in hyperproliferative skin diseases and hyperproliferation associated with wound healing. The expression of elafin is tightly connected with abnormal epithelial differentiation and hyperproliferation, as are keratins 6, 16, and 17. See Wiedow et al, 1991. Elafin is structurally similar to another locally produced antiprotease, secretory leukocyte protease inhibitor (SLPI). Elafin inhibits elastase and proteinase 3, whereas SLPI inhibits elastase and cathepsin G (reviewed by Helmstra, 2002).

The down-regulated genes include alpha 2 actin, actin depolymerizing factor, smooth muscle myosin, myosin light chain kinase, receptor (calcitonin) activity modifying protein 1 (RAMPl), tropomyosin 1, microfibril-associated glycoprotein=2, insulin-like growth factor binding protein 7, and collagen type IV alpha chain.

The greater than two-fold differential expressions of elafin, IL-1RA and RAMP1 genes were confirmed by QC-PCR and Western blot and by immunofluorescent cell staining in fibroblasts cultured from anterior vaginal wall tissues.

MAS5 and RMA algorithms were used to analyze raw data, and the data were normalized using quantiles normalization, then subjected to various statistical analyses (see Example 1 of U.S. patent application Ser. No. 10,684,539). Seventy nine genes were identified by both methods as significant differentially expressed genes. Elafin, keratin 14 and keratin 16 were consistently up-regulated by 16-, 5- and 6-fold, respectively.

These results suggest that both collagen and elastin metabolism may be altered in women with SUI/pelvic floor dysfunction, and that genes in these pathways may be differentially regulated in a hormone-dependent manner. Differential expression of genes related to actin and myosin and certain extracellular matrix genes was observed in studies of pubococcygeus muscle in postmenopausal women with stage III-IV pelvic organ prolapse as compared with women without prolapse (Visco et al, 2003).

2. Modulators of Collagenolysis and Elastolysis The role of gonadal steroids in the etiology of stress incontinence is complex. Both estrogen and progesterone receptors are present in pelvic floor tissue. Current evidence suggests that vaginal tissues from SUI patients may respond differently to estrogen than tissues from asymptomatic controls. Estrogen produces primary effects by directly modulating the expression of MMPs and TIMPs in estrogen-responsive tissues, and secondary effects mediated by growth factors, such as TGF-β. Our studies show that estrogen produces significant increases in TIMP-1 expression in cultured vaginal fibroblasts from continent women without affecting the level and activity of MMP-1, but only minimally affects TIMP-1 expression in cultured SUI vaginal fibroblasts (Chen et al, 2002).

However, there is evidence that estrogen may favor proteolysis in pelvic tissues by growth-factor mediated effects. Estrogen increases circulating levels of TGF-β1 in postmenopausal women with pelvic prolapse, compared to control untreated women (Djurovic et al., 2000). Current data suggests that TGF-β stimulates female pelvic proteolysis. Both TGF-β1 and TGF-β3 inhibit the secretion of MMP-1, an interstitial collagenase, while stimulating the secretion of the elastolytic MMPs, MMP-2 and MMP-9 ((Moalli et al., 2002). TGF-β1 mRNA expression in atrophic endometria from women with pelvic prolapse was increased significantly as compared to women without prolapse (Loverro et al., 1999). TGF-β3 mRNA expression is increased 1.6-fold in vaginal tissue from women with SUI compared to control continent women (FIG. 10 of this application).

The peptide hormone, relaxin, may also modulate collagenolysis and elastolysis in female pelvic support tissues. Relaxin is a peptide with structural homology to insulin and to insulin-like growth factors. It is produced by the female reproductive tract and is found in human corpus luteum and peritoneal fluid from women during the secretory phase of the menstrual cycle. During pregnancy, the level of secretion of this hormone by corpus luteum is increased 20-50 fold. Relaxin is important for the maintenance of pregnancy and the initiation of parturition, as it inhibits uterine contractibility and increases cervix distensibility (Yen S S C, Jaffe R B,1986; Bigazzi et al.,1981; Steinetz et al, 1980), presumably by affecting collagen turnover. Relaxin stimulates the expression of procollagenase mRNA and protein and inhibits TIMP-1 protein expression in cultured human uterine fibroblasts from term pregnancies (Palejwala et al., 2001). The hormone is involved in uterine and cervical growth and remodeling in the pig (Lenhart J A et al., 2001), and in collagen turnover in normal human dermal fibroblasts (Unemori E N and Amento E P, 1990).

The experiments described below were undertaken to investigate the effect of relaxin on elastin proteases and their corresponding inhibitors in pelvic tissues. Specifically, the elastolytic metalloproteinases (MMP-2, MMP-9), the tissue inhibitors of metalloproteinases (TIMP-1, TIMP-2), human neutrophil elastase (HNE) and α-1 antitrypsin (AAT) were compared in SUI and control pelvic fibroblasts. The ability of ATT to inhibit total elastase activity in relaxin-stimulated vaginal fibroblasts was also investigated.

Cultured periurethral vaginal wall fibroblasts from premenopausal SUI and continent women (in both the proliferative and secretory phase of the menstrual cycle) were stimulated with increasing concentrations of relaxin (0-500ng/ml). The secreted proteinases, MMP-2 and M MP-9, were assayed by zymography of conditioned media. The secreted proteinase inhibitors, TIMP-1, TIMP-2, and α-1 antitrypsin (ATT) were evaluated with Western blot. Total elastase activity (cell homogenates, media) was measured by generation of free amino groups from succinylated elastin. Increasing concentrations of ATT were added to cell lysate to evaluate its ability to inhibit total elastase activity.

Immunohistochemical staining of neutrophil elastase, α-1 antitrypsin, and tropoelastin in vaginal wall tissues was performed to confirm the presence of these proteins prior to the fibroblasts culture experiments. The tissues sampled stained for neutrophil elastase, ATT and tropoelastin. Staining for neutrophil elastase, ATT, and tropoelastin was demonstrated in tissues from both phases of the menstrual cycle. Immunofluorescence cell staining confirmed the expression of neutrophil elastase and ATT in pelvic fibroblast cells. The presence of elastin and collagen in the vaginal wall tissue samples was verified with Weigart's and Van Gieson's stains.

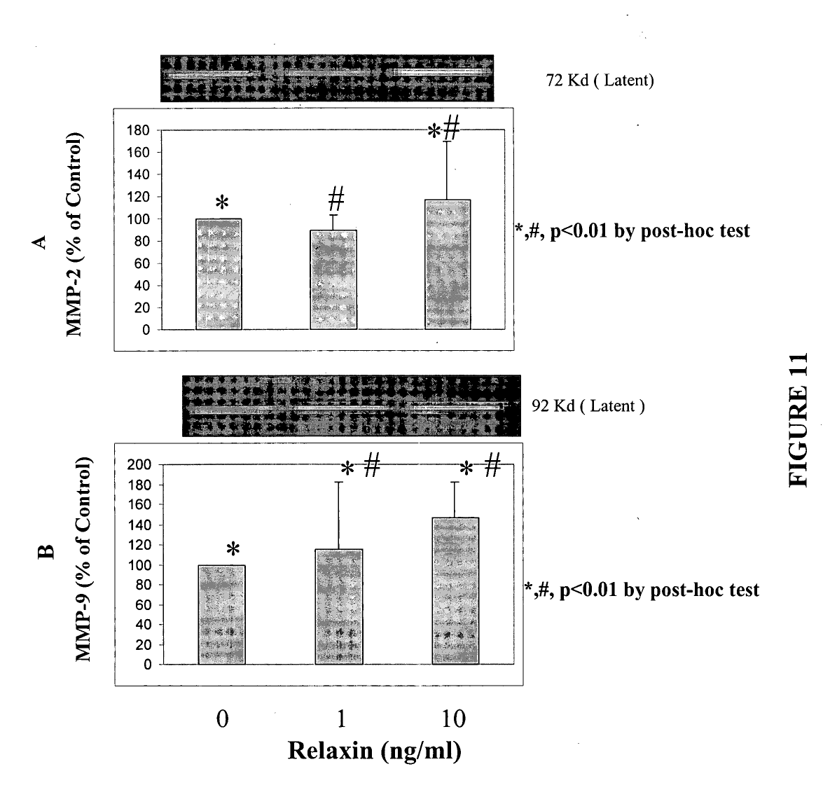

The results of these experiments are shown in FIGS. 11-14 and are summarized in the Table at the end of this section. Additional methodological details are given in the Examples below.

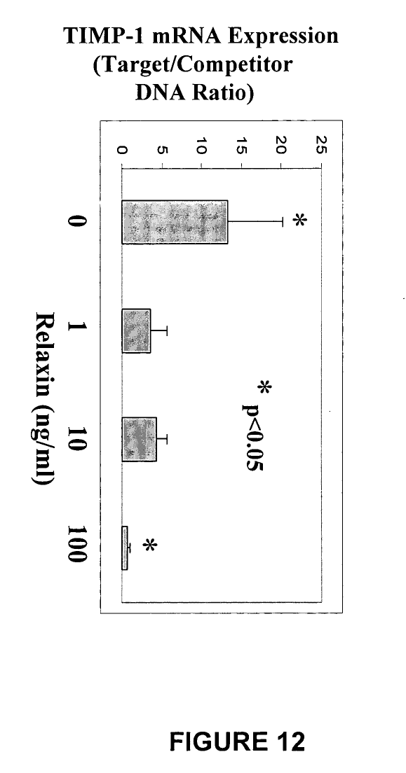

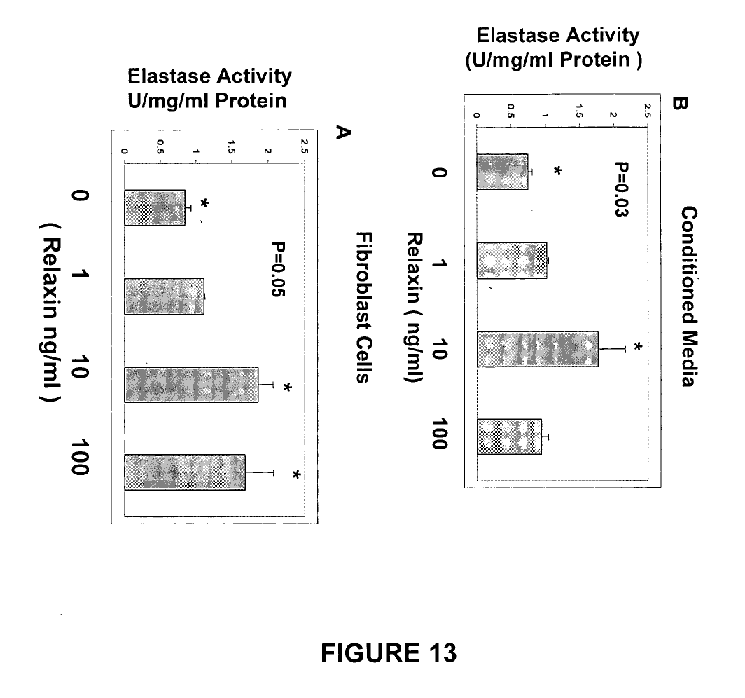

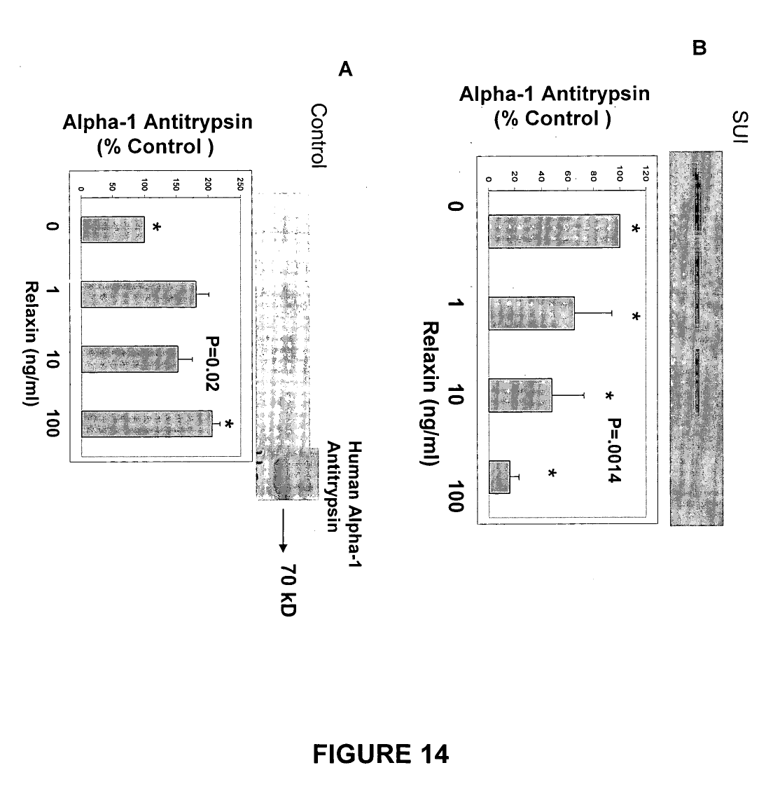

Cultured human vaginal fibroblasts exposed to a physiologic level of relaxin secrete significantly increased amounts of MMP-9 and MMP-2 (FIG. 11) and exhibit decreased TIMP-1 mRNA expression (FIG. 12). Relaxin also elicits significant increases in total elastase activity and α1-antitrypsin protein expression in cultured vaginal fibroblasts and conditioned medium from control, continent women (FIGS. 13 and 14) but decreases α1-antitrypsin expression in SUI vaginal fibroblasts (FIG. 14).

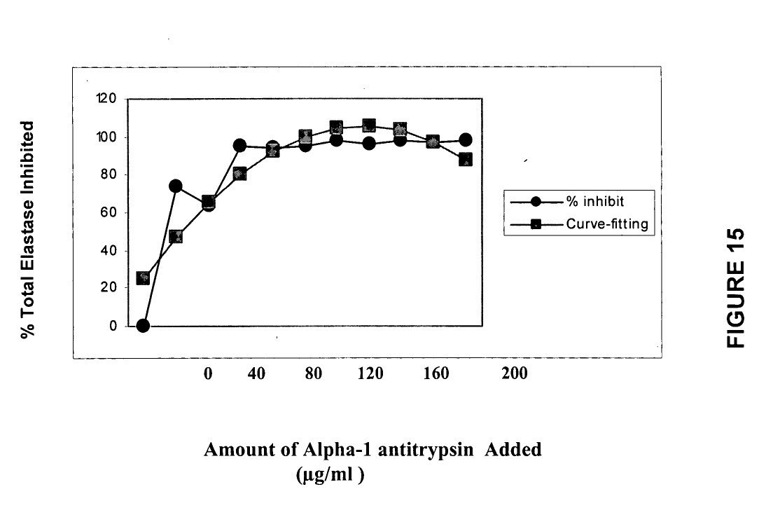

Total elastase activity in cultured SUI vaginal fibroblasts treated with relaxin was inhibited in a dose-dependent manner by ATT, with maximal inhibition of 98% (FIG. 15).

Referring to the Table, proliferative-phase-SUI-fibroblasts demonstrated increases in MMP-2 and no change in MMP-9, TIMP-1, TIMP-2 protein expression with increasing relaxin concentrations. Cells from control women showed increased expression of MMP-2 and MMP-9, but no change in TIMPs. Secretory-phase-SUI fibroblasts showed no response in MMP or TIMP protein expression with relaxin stimulation. Secretory-phase-control fibroblasts reacted by increasing MMP-2, MMP-9, and TIMP-2. With respect to total elastase activity and ATT expression, increasing doses of relaxin appear to increase elastolytic activity in SUI cells by decreasing expression of ATT in proliferative phase cells or increasing total elastase activity in secretory phase cells. Total elastase activity was inhibited in a dose-dependent manner by addition of ATT to SUI cell homogenates .

TABLE
Relaxin modulation of elastases and elastase inhibitors

| Phase of | | | | | | | |
|---|---|---|---|---|---|---|---|
| the | | | | | | Total | |
| Menstrual | Cell | | | | | Elastase | |
| Cycle | Group | MMP-2 | MMP-9 | TIMP-1 | TIMP-2 | Activity | ATT |
| Proliferative | C | + | + | NS | NS | − | − |
| | | p < 0.01 at | p < 0.01 at | | | p < 0.01 at | p < 0.01 at |
| | | 100 ng/ml | 1,10,100 ng/ml | | | 1,10,100 ng/ml | 10 ng/ml |
| | SUI | + | NS | NS | NS | NS | − |
| | | p < 0.04 at | | | | | p < 0.03 at |
| | | 1,100 ng/ml | | | | | 1,10,100 ng/ml |
| Secretory | C | + | + | NS | + | + | + |
| | | p < 0.01 at 10 ng/ml | p < 0.01 at 500 ng/ml | | p < 0.03 at | p < 0.01 at | p < 0.01 at |
| | | | | | 500 ng/ml | 1.10 ng/ml | 100 ng/ml |
| | SUI | NS | NS | NS | NS | + | NS |
| | | | | | | p < 0.04 at 10,100 ng/ml | |
| Legend: | | | | | | | |
| MMP-2, MMP-9, TIMP-1, TIMP-2, total elastase activity and ATT protein expression in pelvic fibroblasts cultured with increasing relaxin concentrations (0-500 ng/ml). Each square represents one dose response curve. Post-hoc analysis (Tukey's honestly significant difference) was applied to | | | | | | | |
| # determine the significance of differences between the means of each relaxin-stimulated cell group and the unstimulated control group. P < 0.05 was accepted as statistically significant. | | | | | | | |

+ indicates a positive dose response (increased expression with increasing relaxin concentrations);
− indicates a negative dose response (decreased expression with increasing relaxin concentrations);
NS indicates no change in response with increasing relaxin concentrations;
C = control cells (continent);
SUI = cells from females with stress urinary incontinence.

2. Methods of Diagnosis and Treatment of SUI and Proteolytic Disorders Affecting Skin and Other Tissues

The above-described studies provide evidence of increased elastase activity and decreased expression of endogenous elastase inhibitors in periurethral fibroblasts of women with SUI. NE makes up a large portion of the activity. This increased activity may result from post-translational modifications or variation in the structure of the enzyme, as it does not appear that changes in gene expression are responsible. Such changes in activity can be exploited for both diagnostic and therapeutic purposes.

Our studies also suggest that relaxin, a female reproductive hormone, may be an etiological factor in the pathogenesis of SUI. The studies disclosed herein provide evidence that relaxin modulates the expression of elastin-degrading enzymes and endogenous inhibitors of elastase in cultured pelvic fibroblasts. SUI pelvic fibroblasts respond differently to relaxin stimulation than normal pelvic fibroblasts. Overall, increasing concentrations of relaxin appear to tip the balance towards increased elastolytic activity in SUI cells by decreasing expression of ATT in proliferative phase cells and by increasing elastase activity in secretory phase cells without corresponding increases in ATT. By contrast, relaxin stimulates increases in both total elastase and ATT in secretory phase control cells. These differential effects of relaxin on normal and SUI tissues can be used for diagnosing SUI.

The finding that ATT is a potent inhibitor of SUI elastase activity suggests that increasing the level of ATT in pelvic tissues of women with SUI may be an effective method for the therapeutic management of SUI and its prophylaxis in women at risk for developing SUI. Other elastase inhibitors may also be useful for treating this disorder (e.g., elafin, SLIP, and small molecule inhibitors).

As the result of its elastolytic effects, prolonged exposure of pelvic supporting tissues to high levels of relaxin, such as occurs with multiple pregnancies, may predispose women to SUI. It is not yet known whether regulation of relaxin levels is aberrant in women with SUI, but this is a possibility in view of the recent report that serum relaxin concentrations during pregnancy tend to be higher in women with pelvic floor dysfunction compared to asymptomatic women (Harvey et al, 2004). Plasma elastase activity is also elevated in women with SUI and circulating inhibitors of elastase are reduced (Mathrubutham et al, 1999). Thus another potential method for the therapeutic management of SUI, including prophylaxis, is to administer an antagonist of relaxin to pelvic tissues of patients in need of therapy. Methods for screening for relaxin antagonists are disclosed in the Examples below.

Disorders that involve abnormal turnover and/or degradation of connective tissue/ECM components are expected to respond favorably to therapies that can be administered directly to affected tissues and are capable of producing long-lasting effects.

For pelvic floor dysfunction and other pathophysiological conditions which are slowly progressive and involve both environmental and genetic components, the efficacy of treatment will likely depend on the availability of diagnostic methods that can be used in subjects who are clinically asymptomatic but at risk for developing the condition, and the use of treatment methods that are effective in preventing the symptoms from developing or arresting the progression of changes in tissue elastin and collagen metabolism that lead to the development of incontinence and pelvic organ prolapse.

In patients with early-stage clinical symptoms, selecting an appropriate treatment may require assessments of both mechanical dysfunction of tissues and organs and molecular/biochemical profiling of the patient's affected tissues. The results of such tests can be used to design and implement treatment methods that ameliorate acute symptoms and produce long-lasting reversal of the underlying metabolic defects.

In patients exhibiting chronic advanced tissue dysfunction, both surgical/reconstructive procedures and adjunctive modulatory therapies may be appropriate.

Other disorders and conditions are potentially treatable by the methods described in this application, including for example, connective tissue changes associated with skin tissue aging and/or physical trauma.

Methods of Treatment

The present invention provides methods of treatment of SUI in women at risk for developing SUI, and in women with symptoms of SUI. The treatment methods may optionally be combined with a method of diagnosis disclosed herein or in U.S. Pat. No. 6,420,119 and U.S. patent application No. 10,684,539, or to other diagnostic methods known to those skilled in the relevant clinical arts.

In one of its aspects, the invention provides a method of treatment comprising delivering an effective amount of an inhibitor of elastase activity to a pelvic support tissue of a patient in need of treatment. The inhibitor may be synthetic inhibitor, preferably a small organic molecule that can be delivered intravaginally (e.g. formulated as a vaginal cream) or to the uterus (e.g., in an intrauterine device). Alternatively, the inhibitor may be an endogenous elastase inhibitor, e.g., alpha-1 antitrypsin (ATT), elafin, secretory leukocyte protease inhibitor(SLPI), alpha 2-macroglobulin and TIMPs, preferably a recombinant DNA sequence encoding an active form of the inhibitor, or a functionally active fragment thereof, which is delivered to the tissue in a genetic construct comprising a promoter operably linked to the DNA sequence and a termination sequence. The genetic construct is preferably designed for stable transformation of the tissue of the patient being treated, although for some purposes, transient expression of the inhibitor may be adequate. The promoter used in the construct may be selected for constitutive, inducible or tissue-specific expression. Many promoters are known in the art that are useful for gene therapy. The DNA coding sequence can be modified to increase the in vivo stability of the expressed polypeptide inhibitor, to facilitate extracellular secretion, and for purposes of detection, provided of course that the modifications do not interfere with the inhibitory activity of the expressed polypeptide. These modifications are well known to those skilled in biotechnology.

The term “functionally active fragment” refers to a truncated sequence which retains a desired biological activity of the full-length sequence. Functionally active fragments of α1-antitrypsin are described, for example, in U.S. Pat. Nos. 6,068,994 and 4,732,973, and in Hercz, (1985), Biochem. Biophys. Res. Commun. 128: 199-203, which are incorporated herein by reference in their entirety. Functionally active portions of TIMPs are also known (see, e.g., references cited in U.S. Patent Publication No. 2003/0073217 A1, which is incorporated herein by reference in its entirety).

Sequences of polynucleotides that are useful in practicing this invention are disclosed in the scientific literature, in public sequence databases such as GenBank, and in the patent literature. See, e.g., WO 0308901 A1 and U.S. Patent Publication No. 2003/0073217 A1, which are incorporated herein by reference in their entirety.

Methods for constructing and using vectors are well known in the art and are described generally in Berger and Kimmel, Guide to Molecular Cloning Techniques, Meth. Enzymology, vol. 152, Academic Press, Inc., San Diego, Calif.; Sambrook et al., (1989) Molecular Cloning-A Laboratory Manual (2^{nd} Ed), vol 1-3, Cold Spring Harbor Laboratory, NY; Current Protocols in Molecular Biology, (F. M. Ausubel et al., eds.) Current Protocols, Greene Publishing Association and John Wiley & Sons, Inc.

Where it is desired to treat patients with multiple antiproteases, fusion proteins comprising different antiprotease sequences can be expressed. See, e.g., U.S. Patent Publication No. 2003/0073217 for methods). Alternatively, two or more genetic constructs, each comprising a different polynucleotide sequence, may be used.

Genetic constructs comprising therapeutic polynucleotides for use in this invention are preferably delivered locally into the affected tissue. Preferably, genetic constructs are injected into the anterior vaginal wall or implanted (e.g., as microparticles) into periurethral tissues. Other possible routes of delivery include topical, intradermal, intramuscular, subcutaneous, and intravascular routes, preferably into blood vessels that supply the tissue to be treated. The expression constructs may be delivered in a non-viral formulation, such as a liposome, a nanocapsule or a microparticle (see, e.g., U.S. Pat. No. 6,627,616) or a liposome modified for in vivo cell-targeted delivery (e.g., Shi and Pardridge, (2000), Proc. Natl. Acad. Sci. USA, 97:7567-7572); Shi et al., (2000), Proc. Natl. Acad. Sci. USA, 98:12754-12759). Alternatively, a replication-defective virus, such as rAAV, may be used. See e.g., International Patent Publication No. WO 03/089011 and references cited therein. For certain tissues, such as muscle, naked DNA can be injected directly into the tissue and is efficiently taken up and expressed. Alternatively, the genetic construct may be transferred into the patient's cells ex vivo and the genetically modified cells reintroduced into the patient's tissues. This last-mentioned approach is potentially useful in patients requiring surgical/reconstructive procedures. Depending on the characteristics of the tissue to be treated, cells may be introduced as such, microencapsulated, bound or adhered to a surface (e.g., a synthetic extracellular matrix, a non-toxic polymer scaffold), or provided in an implantable diffusion chamber.

In another aspect, the invention provides a method of treatment of SUI comprising administering an effective amount of a relaxin antagonist to a pelvic supporting tissue of a patient in need of treatment. The term “relaxin antagonist” as used herein refers to a naturally-occurring or synthetic inhibitor that reduces the ability of relaxin to positively modulate elastolysis. Relaxin antagonists may include, for example, molecules that specifically interfere with the binding of relaxin to cellular receptors or inhibit relaxin signaling pathway(s). Examples of known relaxin antagonists include relaxin receptor fragments and anti-receptor antibodies.

It is contemplated that either or both of the above methods will be effective for prophylactic therapy of patients at high risk for developing SUI. For example, women who have undergone multiple pregnancies can be treated with a relaxin antagonist immediately post-partum, with continued treatment thereafter. Women with a family history of SUI may be good candidates for gene-based therapy.

The invention also encompasses methods of treatment of SUI by downregulating or silencing genes that modulate elastolysis by activating elastases, particularly SUI neutrophil elastase. In this regard, antisense oligonucleotides, ribozymes and siRNA are potentially useful.

The above treatment agents are administered to the patient in a pharmaceutical composition in a therapeutically effective amount. Pharmaceutically acceptible carriers are well known in the art and are described, for example, in Remington's Pharmaceutical Sciences. A therapeutically effective amount is an amount that is effective for treating the disorder. Optimal dose ranges can be assessed by in vitro assays and in animal models. The dose required for administration to an individual patient depends on the condition being treated, the age and condition of the patient and the route of administration, and is best decided by a skilled clinician.

Diagnostic Tests

In another of its aspects, the invention provides a diagnostic test for SUI which is based on altered responses of SUI pelvic tissue fibroblasts to relaxin as compared to normal cells.

In one embodiment, the test comprises incubating cultured pelvic fibroblasts from a test subject in the presence and absence of relaxin and comparing the level of ATT protein and total elastase activity in relaxin-treated and untreated cells, wherein a change in either one of the level of ATT or total elastase activity, but not both, is indicative of SUI, and wherein a unidirectional change in the level of both ATT and total elastase is indicative of normalcy.

In another embodiment, the method additionally comprises comparing one or more of MMP-2, MMP-9 and TIMP-2 protein expression in the presence and absence of relaxin in test cells as compared with normal cells.

The invention also encompasses in vitro assays for identifying modulators of elastolysis and relaxin antagonists.

### EXAMPLES

These Examples are presented to illustrate the practice of various embodiments of the invention and are not intended to limit the scope of the invention as claimed.

### Example 1.

### Analysis of Expression of Elastase and Elastase Inhibitors in Tissues

The use of quantitative competitive PCR to quantitate levels of expression of elastases and elastase inhibitors in a tissue is illustrated below for human neutrophil elastase, cathepsin K, alpha-1- antitrypsin and TIMP-1.

Tissues for analysis are excised from patients and immediately frozen in liquid nitrogen. Total RNA is extracted from the frozen tissues using the guanidium isothiocyanate method (RNAzol, Tel-test Inc, Friendswood, Tex.). The amount and purity of RNA is quantitated by spectrophotometry in a GenQuant RNA/DNA calculator (Pharmacia Biotech, Cambridge, UK).

Specific sequences of oligonucleotide primers for human neutrophil elastase, MMP-9, alpha-1- antitrypsin, elafin, and TIMP-1 were obtained from Gene Bank Database of the National Center for Biotechnology Information (NCBI) of the National Institutes of Health, or the biological literature (cathepsin K). Corresponding sets of primers for each of these oligonucleotide primers was found with the help of the program OLIGO 5.0 Primer Analysis Software (National Bioscience, Plymouth, Minn.) and synthesized by the “Protein, Aminoacid and Nucleicacid (PAN)-Facility,” Beckman Center, Stanford University, Stanford, Calif. Oligonucleotide sequences are shown below for cathepsin K, alpha-1-antitrypsin and human neutrophil elastase, and TIMP-1.

| | | | | Primer |
|---|---|---|---|---|
| | | | | Location |
| | Primer | | Size | on mRNA |
| mRNA | 5′-3′ | Primer Sequence | (bp) | SEQ |

| | | | | |
|---|---|---|---|---|
| Cathepsin | 5′-end | AGCTGGGGAGAAAACTGGGG (SEQ ID NO: 1) | 253 | 1000 |
| K | 3′-end | TGAAGCACAAACAAATGGGGAAACTGAACA | | 1223 |
| | | (SEQ ID NO: 2) | | |
| | 3′-end | TGAAGCACAAACAAATGGGGAAACTGAACAGGCTGG | 144 | 1200 |
| | floating | CTGGAGTG (SEQ ID NO: 3) | | |
| Alpha-1 | 5′-end | TGCCCAGAAGACAGATACAT (SEQ ID NO: 4) | 647 | 112 |
| antitrypsin | 3′-end | GCTTCATCATAGGCACCTT (SEQ ID NO: 5) | | 741 |
| | 3′-end | GCTTCATCATAGGCACCTTGGTGGTCAGCTGGAG | 341 | 419 |
| | floating | (SEQ ID NO: 6) | | |
| Human | 5′-end | CGTGGCCCTTCATGGTGTC (SEQ ID NO: 7) | 580 | 157 |
| Neutrophil | 3′-end | GCAAAGGCATCGGGGTAGAG (SEQ ID NO: 8) | | 717 |
| Elastase | 3′-end | GCAAAGGCATCGGGGTAGAGCGGCGTCCCTGAG | 333 | 457 |
| | floating | SEQ ID NO: 9) | | |
| TIMP-1 | 5′-end | TTC CAC AGG TCC CAC AAC CGC AGC (SEQ ID NO: 10) | 228 | 346 |
| | 3′-end | CGT CCA GCA ATG AGT (SEQ ID NO: 11) | | 556 |
| | 3′-end | CGT CCA GCA ATG AGT GGC TGT TCC AGG GA | 106 | 346 |
| | floating | (SEQ ID NO: 12) | | |

One μg total RNA from each tissue sample was reverse transcribed to cRNA using the Gen Amp RNA PCR kit (Perkin-Elmer, Foster City, Calif.) prior to performing PCR, in order to amplify the cDNA, as previously described (Chen et al, 2002).

Competitive and target- cDNA fragments were constructed as follows. A 647 base pair (bp) fragment of native alpha-1 antitrypsin cDNA, a 580 bp fragment of native HNE cDNA, a 253 bp fragment of cathepsin K cDNA and a 288 bp fragment of native TIMP-1 cDNA (i.e., the target) was obtained by PCR amplification of reverse-transcribed total RNA from the tissue with the regular 3′ and 5′ primers. The PCR product was visualized by agarose gel electrophoresis stained with ethidium bromide (ETB). The cDNA was extracted from the gel, purified with an agarose gel extraction kit (Amersham Pharmacia Biotech Ltd., Piscataway, N.J. ), and quantitated by spectrophotometry (Pharnacia Biotech Ltd., Cambridge, U K). T o construct a competitive c DNA fragment, a 3′ floating primer with a sequence complementary to the cDNA between the 3′ and 5′ primer binding sites was designed by attaching the complementary sequence of the binding site of the original 3′-end alpha-1 antitrypsin , HNE, cathepsin K or TIMP-1 primer. After PCR with the regular 5′-end primer and the 3′-end floating primer, the PCR product was visualized by agarose gel electrophoresis stained with ETB, and cDNA extraction, purification, and determination of the concentration were performed as described above. This step resulted in cDNA fragments of alpha-1 antitrypsin (341 bp), HNE (333 bp), cathepsin K (144 bp) and TIMP-1 (124 bp). Independent sequence analysis was performed to confirm the identity of the expected sequence and amplified cDNA.

Quantitative competitive RT-PCR was carried out by constructing a standard curve for alpha-1 antitrypsin, HNE, cathepsin K and TIMP-1 by co-amplification of a constant amount of competitive cDNA (0.0001, 0.000001, 10 attomol and 0.1 attomol, respectively) with declining amounts of target cDNA (0.3-0.000625 fmol) obtained by serial dilution. Sixty microliters of the cDNA mix were added to 40 μL PCR-Mastermix containing 1.9 mmol/L MgCl₂ solution, 10×PCR buffer II, 0.2 mmol/L of each deoxy-NTP, 2.5 U Taq polymerase (Perkin-Elmer Corp, Foster City, Calif.), corresponding paired primers in a concentration of 0.2 μmol/L of each primer to a total volume of 100 μL, and 14.5 μL DEPC-treated H₂O. The reaction was covered with 50 μL light white mineral oil and put in the Perkin-Elmer Corp. DNA Thermal Cycler 480. PCR cycles were started at 95° C. for 5 min to denature all proteins; 30 cycles for 45 s at 94° C.; 45 s at 55° C. (alpha-1 trypsin) or 60° C. (Cathepsin K); 65° C. (HNE, 40 cycles); 60 s at 72° C. The reaction was terminated at 72° C. for 5 min and was quenched at 4° C. Two percent agarose gel (Life Technologies, Inc. Gaithersburg, Md.) electrophoresis was carried out. The gel blot was analyzed by UV densitometry (Gel-Doc 1000 system, Bio-Rad Laboratories, Inc., Hercules, Calif.). The logarithmically transformed ratios of target cDNA to competitive cDNA were plotted against the log amount of initially added target cDNA in each PCR to obtain the standard curve. This standard curve was highly reproducible and linear. The values obtained from the regression line of the standard curve (y=b+mx) allowed us to calculate the amount of cDNA transcripts in an unknown sample. Competitive cDNA were added to each unknown sample before PCR (i.e., 0.0001 attomol alpha-1 antitrypsin, 0.000001 pmole HNE, 10 attmol cathepsin K and 1 fmol TIMP-1). The ratios of the densities of sample target cDNA to competitive cDNA for each of these molecules were logarithmically transformed and compared with the values obtained from standard curve. Because the efficiency of the amplification of the internal control molecule (the competitive cDNA) is identical to that of the target template, quantitative PCR avoids discrepancies associated with tube-to-tube or sample-to-sample variations in the kinetics of the RT reaction.

The level of mRNA expression of an elastase enzyme relative to the MRNA expression of an inhibitor in the tissue being analyzed can be expressed as a ratio. The ratio can be compared to a predetermined indicator to determine whether the ratio is outside the normal range. The predetermined indicator is typically based on statistical assessment of expression ratios obtained from normal subjects and subjects with a disorder or condition in which elastin metabolism is known or suspected to play an important role. Ratios can be determined from quantitative measurements of either or both mRNA and protein levels or activities.

### Example 2.

### Measurement of Elastolytic Activity

Tissues were cut into small pieces and homogenized in 0.5 ml solubilized buffer (150 mM NaCl, 1% N-40, 0.5% deoxycholate, 0.1% SDS, 4 mM EDTA, 50 mM Tris-HCl, 2 mM PMSF, pH 7.4), then transferred into small tubes and rotated at 4° C. overnight. Solubilized protein was collected after centrifugation at 10,000 g for 30 minutes. Protein concentrations were determined by Protein Assay Kit (Bio-Rad, Hercules, Calif.).

Elastolytic activity in the cell homogenate (or secreted into the culture medium) was determined by the generation of free amino groups from succinylated elastin, according to Rao et al (Anal. Biochem. 250: 222-227 (1997). A chemically modified porcine elastin (Sigma) with succinylated amino groups (Sigma) was used as a substrate. Briefly, 50 μl of enzyme sample was added to 100 μg succinylated elastin in 50 μM sodium borate buffer (pH 8.0, Sigma) and incubated at 37 ° C. for 1 hour. Fifty μl of a 0.03% solution of TNBSA (Sigma) was added to each reaction and left for 20 minutes at room temperature. The optical density of each reaction was determined using Molecular Devices microplate reader at 450 nm. Porcine elastase (CalBiochem, La Jolla, Calif.) was used to make a standard curve. The elastolytic activity was normalized for protein concentration.

### Example 3.

### Measurement of Collagenolytic Activity in a Tissue

Collagenase activity is assessed by extracting total collagen from the tissue and quantitating the level of the carboxy-terminal neoepitope by ELISA. See e.g., Billinghurst R C et al., J Clin Invest 1997;99:1534-1545.

### Example 4.

### Zymography of Secreted Proteinases

Zymography of secreted proteinases in conditioned media derived from cultured fibroblast cells was carried out by electrophoresis in 10% SDS-polyacrylamide gels containing 0.1% gelatin. Gels were then soaked for 40 min. in 2.5% Triton X-100, incubated 12 hr. at 37° C. in 0.05 M Tris, pH 8, 5 mM calcium chloride, 0.02% azide, and stained with 1% Coomassie blue R-250, 25% ethanol, and 15% formaldehyde. HT1080 tumor cell supernatant is used as a positive control for enzymatic activity. Three to five separate zymograms were performed on the supernatants from each set of relaxin-stimulated fibroblasts to confirm reproducibility of the assays.

### Example 5.

### Western Blot Analysis

Supernatant TIMP-1, TIMP-2, and ATT expressions were assessed semi-quantitatively by Western-blot. Five micrograms of total protein from conditioned media were separated by 10% SDS-PAGE under reducing conditions and blotted onto Nitrocellulose Membranes (Pierce, Rockford, Ill.) in an electrophoretic transfer cell (Bio-Rad, Hercules, Calif.). Blots were blocked with 5% non-fat milk in Tris-HCl with Tween (TBS-T) at 4° C. overnight. Mouse anti-human TIMP-1 primary antibody (1 μg/ml, Oncogene, Boston, Mass.) or TIMP-2 primary antibody (5 μg/ml, Oncogene, Boston, Mass.) and sheep anti-mouse IgG-HRP conjugated secondary antibody ( 1/10,000, Amersham, Buckinghamshire, England) were diluted in TBS-T. Rabbit anti-human ATT primary antibody ( 1/5000, Sigma) and donkey anti-rabbit IgG-HRP conjugated secondary antibody ( 1/5000, Amersham, Buckinghamshire, England) were diluted in TBS-T. Blots were developed by chemiluminescence. Densitometry of immunoreactive bands on Western blot was performed with Molecular Analyst Software (Bio-Rad). At least three separate Western blot experiments were carried out on the supernatants from each set of relaxin-stimulated fibroblast cultures to confirm reproducibility of our assays.

### Example 6.

### Immunohistochemical Staining

Immunohistochemical staining for neutrophil elastase, ATT, and tropoelastin was performed to confirm the presence of these proteins in vaginal wall tissues. The vaginal tissues were fixed with 4% paraformaldehyde then embedded in paraffin, sectioned, and mounted. Twenty serial sections (5 μm) from each sample were then prepared for immunohistochemistry, and the first and last sections were stained with hematoxylin-eosin and examined with a Nikon microphot-FXA microscope (Nikon Instruments, Garden City, N.Y.). Sections were deparaffinized with xylene and quickly rehydrated through graded alcohols. Excess liquid was removed, and sections were washed with TBS-T (pH 7.4, 0.02% Tween-20). In order to block unspecific binding, sections were pre-incubated in humidity chambers for 30 min at room temperature in TBS-T containing 5% normal goat serum (Sigma Chemical Co., St. Louis, Mo.) and 1% BSA. After washing in TBS-T, three times for 5 min each, sections were incubated over night at 4° C. with rabbit anti-human α-1 antitrypsin antibody at a dilution of 1/20 (Sigma) or rabbit anti-human neutrophil elastase at a dilution of 1/50 (Research Diagnostics, Inc. Flanders, N.J.) or rabbit anti-human tropoelastin at a dilution of 1/50 (Elastin Products Company, Owensville, Mo.). After washing in TBS-T for 3 times, sections were incubated with a secondary antibody, biotinylated goat anti-rabbit IgG (dilution 1/50; Sigma Chemical Co.), in humidity chambers for 60 min at room temperature. Negative controls were incubated with TBS-T containing 5% goat serum and 1% BSA without primary antibody. To amplify the signal, sections were washed with TBS-T, and then the avidin-biotin alkaline phosphatase-staining method (Vector Lab., Inc., Burlingame, Calif.) was used. Endogenous alkaline phosphatase activity was inhibited by the addition of levamisole to the buffer used to prepare the substrate solution. Finally, slides were c ounterstained with 25% hematoxylin, cleared, cover slipped, and examined by a Nikon DX-DB2 camera and a Nikon microphot-FXA microscope. A red precipitate indicated positive staining by the primary antibody.

### Example 7.

### Immunofluorescence Cell Staining

Fibroblasts from vaginal cuff were cultured in a 4-well chamber slide. The cells were fixed with 4% PFA and treated with 5% Triton. After washing with TBS-T and blocking with 5% normal goat serum and 1% BSA, the slides were incubated with 1/50 rabbit anti-human leucocyte elastase (RDI, Flanders, N.J.) or mouse anti-vimentin (Chemicon, Temecula, Calif.) or 1/200 rabbit anti-human α-1 antitrypsin primary antibody at 4° C. overnight. The primary antibody was not added in the negative control. After washing, the slides were incubated with goat anti-mouse IgG-TRITC (vimentin) and goat anti-rabbit-IgG-FITC (α-1 antitrypsin) or goat anti-rabbit-IgG-FITC (neutrophil elastase) at room temperature for 1 hour in a dark chamber. The slides were then examined with a fluorescence microscope after washing and mounting.

### Example 8.

### Elastin and Collagen Stain

Weigert's solution was used to stain elastin and Van Gieson's mixture of picric acid and acid fuchsin was used to stain collagen as described in Drury R A B, Wallington E A., Carleton's Histological Technique. London, England: Oxford University Press, 1976. Vaginal tissues were fixed and sectioned using the same method as described above in Example 6. These were deparaffinized with xylene and quickly rehydrated through graded alcohols. They were then placed into Weigert's solution for 40 minutes at room temperature and washed with 95% ethanol to remove excess solution and differentiated with 1% acid alcohol. The nuclei were then stained with Hematoxylin and elastin with Van Gieson's solution.

### Example 9.

### In Vitro Screening Assays for Candidate Modulators of Elastolysis in Pelvic Supporting Tissues

In vitro cell based assays are useful in screening for candidate modulators of elastolysis in pelvic supporting tissues. Knowledge of differentially expressed genes in pelvic supporting tissue of continent and incontinent females provides a useful starting point for identifying candidate modulators for screening. This Example illustrates the use of a cell-based assay to identify relaxin as a modulator of elastolysis in pelvic fibroblasts.

Tissue Collection

After informed consent was obtained, approximately 1 cm² of full-thickness, peri-urethral vaginal wall was excised 1 cm lateral to the urethrovesical junction from premenopausal women undergoing surgery for pelvic floor dysfunction. Smaller, 0.5 cm² biopsies of vaginal wall from a similar area were excised in premenopausal continent, control women undergoing benign gynecologic surgeries. The urethrovesical junction was identified by insertion of a Foley balloon in all participants. The area lateral to this junction was infiltrated with saline and a vaginal wall incision made with the scapel. Full thickness vaginal wall biopsies were taken after sharp dissection down to the avascular space of loose areolar tissue.

Fibroblast Cell Culture

Fibroblast explants were cultured by cutting tissue samples into approximately 1 mm³ fragments and placing 10 fragments into a 25 cm² tissue culture flask for primary explantation. Tissue fragments were allowed to attach to the plastic surface for 15 minutes. Ten milliliters of culture medium, which consisted of 90% DMEM and 10% fetal bovine serum, were then added to each disk. Cultures were incubated at 37° C. in an atmosphere of 95% air and 5% C02. These cultured cells were confirmed as fibroblasts free of contaminating epithelial cells by immunohistochemistry of the specific marker, Vimentin. Cells were grown to confluence (10-12 weeks) and were utilized up to the second passage. After confluence, cells were cultured in serum free DMEM with 0.2% lactalbumin hydrolate (LAH) for 72 hours. Medium was removed and replaced with or without various physiologic concentration levels of human relaxin (0-500 ng/ml) for 48 hours. The medium from each culture (“culture supernatant”) was collected and concentrated by 100 fold with Centricon. The cells were harvested with cold PBS containing 0.02% Triton X-100. The supernatant from the different relaxin-concentration stimulated cells was used to determine total elastase activity. The supernatant was also used to evaluate MMP-2 and MMP-9 by zymography, and TIMP-1, TIMP-2, and ATT by Western blot. The cell homogenate from the fibroblasts stimulated with 100 ng/ml of relaxin was used to investigate α-1 antitrypsin inhibition of elastase activity in these cells.

Tests for Antagonists of Relaxin

Secretory phase fibroblasts are preferably used to screen for antagonists of relaxin effects on elastolysis. The fibroblasts are incubated with a stimulatory concentration of relaxin alone, the candidate antagonist alone, and relaxin and the candidate antagonist together. Typically, several concentrations of the candidate antagonist are tested, and the test compound is added to the cultures prior to or at the same time as relaxin. Cytotoxicity assays can be performed to determine whether putative antagonists identified in this assay produce cell toxicity. Compounds that antagonize the effects of relaxin are further tested in competition binding experiments with relaxin receptors to identify putative receptor antagonists.

Tests for Putative Modulators

Putative modulators of elastin metabolism in pelvic fibroblasts include, for example, cytokines, hormones and growth factors. Time course studies of both mRNA and protein expression of elastolytic and collagenolytic enzymes and their endogenous inhibitors in fibroblast cell cultures are carried out to determine an appropriate time frame for these experiments, which is expected to be in the range of 24-48 hours. Dose response studies are carried out with each putative modulator, and combinations thereof.

After 24 hours of incubation, culture supernatants are isolated and concentrated and fibroblast monolayers are homogenized using Triton X-100. Total elastase activity is measured, and elastase activities in supernatants and cell homogenates are examined by gel electrophoresis with zymography and Western blots as described herein to identify the type(s) of elastase enzymes and inhibitors whose activity is modulated. Replicate cultures are harvested for analysis of mRNA transcripts by QC-RT-PCR as described elsewhere in this application.

### Example 10.

### Treatment of Aging Human Skin

Changes in elastin degradation in aging skin may be produced by mechanisms similar to those that lead to stress urinary incontinence. A decline in the ratio of alpha-1-antitrypsin protein expression to total elastase activity in skin of postmenopausal women with decreased estrogen, as compared with premenopausal women, may be a predisposing factor in both of these conditions. Such a change may result from a decrease in the level of active alpha-1-antitrypsin, with or without changes in levels of tissue elastase activity.

Measurements of alpha-1-antitrypsin protein expression and elastase activity will be made in full thickness skin after removal of fat from the underside of the dermis. Skin will be taken from young women (25-35 years of age) at the time of routine pelvic surgery. The phase of the cycle, proliferative or secretory, will be noted. Comparable tissue will also be taken from post-menopausal women not replaced with estrogen at the time of pelvic surgery for benign lesions. This tissue will be treated in the same way as tissues from younger women. Facial skin, obtained from women undergoing cosmetic surgery for wrinkles and excess skin, will also be assayed for the ratio of alpha-1-antitrypsin to elastase activity.

The detection of imbalances in the ratio of alpha 1 antitrypsin to elastase will provide a basis for designing a treatment method, which can be further tested in cultured tissues and in animal models.

As presently envisaged, a method of treatment of tissues having aberrant ratios of alpha-1 antitrypsin to total elastase may involve administering a genetic construct comprising a polynucleotide sequence(s) (or a functional fragment thereof) to the patient for expression in the affected area(s). The polynucleotide sequence or sequences should be capable of inhibiting endogenous elastolytic activity when expressed. The expression should, be sufficient to reverse the degradative process, and the duration of the effect should be long-lasting. For example, the procedure may involve ex vivo transformation of fibroblasts, cultured from pelvic tissue and/or facial tissue of the patient, with a vector comprising the alpha-1-antitrypsin coding sequence. The expression of alpha-1-antitrypsin MRNA and protein by the transformed cells is monitored using QC-PCR and Western blot analysis. The fibroblasts are reintroduced into the patient. An alternative procedure is to use an in vivo transformation procedure as described elsewhere in this application.

Current treatments for reduction of wrinkles in aging and/or damaged skin include injection of Botox, microderm-abrasian, collagen injection and the injection of dermal fillers, such as hyaluronic acid. Cosmetic compositions containing serine protease inhibitors are disclosed and claimed in U.S. Pat. No. 6, 294,181. It has been suggested that exposure of skin to pulses of light at specific frequencies may be capable of stimulating the production of new collagen and inhibiting collagen breakdown. It is believed that none of the foregoing treatments is capable of producing long-lasting effects, however.

The methods of treatment disclosed in this Example and elsewhere in this application are believed to be applicable to the treatment of SUI and other disorders involving aberrant extracellular matrix metabolism.

### Example 11.

### Analysis of Differential Gene Expression in Pelvic Supporting Tissue Using Oligonucleotide Microarrays

Patient Selection

Subjects were premenopausal women undergoing benign gynecologic surgery with no history of endometriosis, gynecologic malignancies, pelvic inflammatory conditions, connective tissue disorders, emphysema or prior pelvic surgery. Continent women were considered as controls while women undergoing surgery for urinary incontinence were identified as cases. The diagnosis of urinary incontinence was confirmed by urodynamic studies. The subjects were matched for age, parity and body mass index. The degree of pelvic organ prolapse in both groups of subjects was no greater than stage I as determined by POP-Q. The study was approved by the Institutional Review Board of Stanford University Medical School. Informed consent of the subjects was obtained for excision of pelvic supporting tissue used in this study.

Tissue Collection and RNA Isolation

Approximately 1 cm² of full-thickness, peri-urethral vaginal wall was excised 1 cm lateral to the urethrovesical junction identified by a Foley balloon, from women undergoing surgery for stress urinary incontinence. A 0.5 cm² sample of uterosacral ligament was also obtained from six participants for comparison studies between different pelvic tissues. Smaller, 0.5 cm² biopsies of vaginal wall from a similar area were excised in continent, control women undergoing benign gynecologic surgeries. For analysis, tissue samples were selected from subjects in the proliferative (estrogen only) phase of the menstrual cycle and from subjects in the secretory (estrogen plus progesterone) phase of the menstrual cycle. Tissue specimens were frozen in liquid nitrogen immediately after excision.

Total RNA was extracted with TRIZOL reagent (Gibco BRL Life Technologies, Grand Island, N.Y.) according to the protocol suggested by the manufacturer. At least 30 μg total RNA was extracted from the tissue, and a portion was subjected to gel analysis to verify the integrity of the RNA.

Extraction. Amplification, and Labeling of mRNA

Extraction of total RNA, amplification, and labeling of mRNA were carried out as previously described (Mahadaveppa and Warrington, (1999) Nat. Biotechnol. 17 (11): 1134-6) and as published in the GeneChip® Expression Technical Manual (Affymetrix, Inc., Santa Clara, Calif.).

Sample quality was assessed using four criteria: the appearance of 18S and 28S rRNA by agarose gel electrophoresis; an A260/A280 spectrophotometric ratio less than or equal to 2; a GAPDH (glyceraldehyde-3-phosphate dehydrogenase) 3 prime to 5 prime ratio less than 3; and a minimum of 26% transcripts detected as present calls in each sample.

Labeled target was fragmented by incubation at 94 ° C. for 35 min in the presence of 40 mM Tris-acetate, pH 8.1, 100 mM potassium acetate, and 30 mM magnesium acetate. The hybridization solution consisted of 20 μg fragmented cRNA and 0.1 mg/ml sonicated herring sperm DNA in 1× MES buffer (containing 100 mM MES, 1M Na+, 20 mM EDTA, and 0.01% Tween 20).

Hybridization, subsequent washing, and staining of the arrays were carried out as outlined in the GeneChip® Expression Technical Manual (Affymetrix, Inc) on Affymetrix arrays. HuGene F1 arrays were used for analysis of gene expression in proliferative phase tissue samples and Human Genome U133A oligonucleotide chip sets were used for analysis of gene expression of secretory phase tissue samples. The arrays were synthesized as described previously using light-directed combinatorial chemistry (Fodor et al., (1993) Nature 364 (6437): 555-6), which is incorporated herein by reference).

Following washing and staining, probe arrays were scanned twice (multiple image scan) at 3 μm resolution using the GeneChip® System confocal scanner made for Affymetrix by Hewlett-Packard, Inc., Palo Alto, Calif.) and GeneChip® Scanner 3000.

Microarray Data Analysis

GeneChip® 5.0 Software (Affymetrix, Inc.) was used to analyze the scanned images and to obtain probe usage and quantitative information. The images were analyzed to determine an intensity value for each probe set within each gene represented on the array. The MAS 5 default algorithm (Affymetrix Microarray suite 5.1©) defines an average difference for each probe set on each array using a log₂ transformed probe intensities to correct for nonspecific binding. For analysis of data generated with U133A arrays, in addition to the MAS 5 algorithm, we used the Robust Multiarray Average (RMA) algorithm developed by Rafael Irizarray (Department of Biostatistics, Johns Hopkins University) for background correction and quantile normalization for normalizing the probe intensities. These algorithms are available in microarray analysis packages Bioconductor of the open source statistical software R, http://www.r-project.org.

Microarray data was subjected to statistical analysis using multiple methods to identify genes that were differentially expressed in SUI subjects as compared with continent controls. These methods included: simple t tests of both parametric and non-parametric formulations, PAM (Prediction Analysis for Microarrays, Tibshirani et al, (2002) Proc Natl Acad Sci USA 99: 6567-6572). The calculated p values were then adjusted according to different multiple testing procedures and cut-off points were set to select genes that showed significant differences in differential expression at different levels. A common list of significant genes was then generated from these methods.

Hierarchical Clustering

Hierarchical clustering analysis was performed with data obtained from tissue samples taken during the estrogen phase of the menstrual cycle to assess the ability of the 90 up- and down-regulated candidate genes in SUI to discriminate between normal and affected individuals. A matrix based ward clustering analysis employing the Cosine correlation of similarity coefficient was performed using GeneMaths software (Applied Maths, Kortrijk, Belgium). The results obtained suggest that the analyzed genes may be important for distinguishing SUI and control pelvic supporting tissue phenotypes and may reflect complex genetic variations which may be predictive of the development of incontinence in individuals who have not yet developed the clinical symptoms of this condition.

In like manner, microarray expression profiling of various tissues can be used to identify genes that are differentially expressed in other connective tissue disorders.

### Example 12.

### Western Blot Analysis of Elafin, Cathepsin K and Alpha 1-Antitrypsin

Twenty micrograms of total protein from each patient was separated by 10% SDS-PAGE under reducing conditions and blotted onto nitrocellulose membranes (Pierce, Rockford, Ill.) in an electrophoretic transfer cell (Bio-Rad, Hercules, Calif.) for elafin determination. One hundred micrograms of total protein from each patient was electrophoresed for cathepsin K and alpha-1 antitrypsin. Blots were blocked with 5% non-fat milk at 4° C. overnight. After blocking, the membrane was washed three times in PBST (PBS, pH 7.4 and 0.02% Tween. The membrane was incubated in 1:100 dilution of rabbit polyclonal antibody to human elafin (SKALP) (Cell Science, Norwood, Mass.), 1:100 dilution of goat anti-human cathepsin K (Santa Cruz Biotechnology, Inc., Santa Cruz, Calif.) or 1:5000 dilution of rabbit anti-human alpha-1 antitrypsin (Sigma, St. Louis, Mo.) for 1 hour at room temperature, followed by 3 washes in PBST. The membrane was then incubated in 1:5000 dilution of donkey anti-rabbit IgG or 1:100,000 dilution of mouse anti-goat/sheep IgG conjugated to HRP for 1 hour at room temperature, followed by three washes in PBST. Blots were developed by chemiluminescence.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the invention disclosed in this application.

All of the publications, patent applications and patents cited in this application are herein incorporated by reference in their entirety to the same extent as if each individual publication, patent application or patent was specifically and individually indicated to be incorporated by reference in its entirety.

SEQ ID NOs: 1-12 are set out in the attached Sequence Listing. The codes for polynucleotide and polypeptide sequences used in the attached Sequence Listing confirm to WIPO Standard ST.25 (1988), Appendix 2.

### References

Olsen A L, Smith F J, Bergstrom J O, Colling J C, Clark A L. Epidemiology of surgically managed pelvic organ prolapse and urinary incontinence. Obstet Gynecol 1997;89(4):501-6.
Sampselle C M, Harlow S D, Skurnick J, Brubaker L, Bondarenko I. Urinary incontinence predictors and life impact in ethnically diverse perimenopausal women. Obstet Gynecol 2002;100(6):1230-8.
Howard D, Delancey J O, Tunn R, Ashton-Miller J A. Racial differences in the structure and function of the stress urinary continence mechanism. Obstet Gynecol 2000;95(5):713-17.
Rortveit G, Daltveit A K, Hannestad Y S, Hunkskaar S. Urinary incontinence after vaginal delivery or cesarean section. The New England J of Medicine 2003 ;348(10):900-7
Prosser I W and Mecham R P. Regulation of extracellular matrix accumulation: A short review of elastin biosynthesis. In: Varner J E, ed. Self-assembling architecture. New York: Alan R. Liss, 1988. p. 1-23.
Finlay G A, O'Donnell M D, O'Connor C M, Hayes J P, FitzGerald M X. Elastin and collagen remodeling in emphysema. A scanning electron microscopy study. Am J Pathol 1996;149:1405-15.
Yamamoto K, Yamamoto M, Akazawa K, Tajima S, Wakimoto H, Aoyagi M. Decrease in elastin gene expression and protein synthesis in fibroblasts derived from cardinal ligaments of patients with prolapsus uteri. Cell Biol Int. 1997;21(9):605-11.
Chen B H, Wen Y, Zhang Z, Wang H, Warrington J A, Polan M L. Menstrual phase dependent gene expression differences in periurethral vaginal tissue from stress incontinent women. Am J Ob Gyn 2003;189(1):89-97.
Chen B H, Wen Y, Polan M L. Elastolytic activity in women with stress urinary incontinence and pelvic organ prolapse. Neurourology and Urodynamics 2004;23(2):119-26.
Chen B H, Wen Y., Li H, Polan M L. Collagen metabolism and turnover in women with stress urinary incontinence and pelvic prolapse. Int. Urogynecol J 2002; 13: 80-87.
Visco A G and Yuan L. Differential gene expression in pubococcygeus muscle from patients with pelvic organ prolapse: Am J. Obstet. Gynecol. 2003; 189: 102-12.
Werb Z B M, McKerrow J H, Sandhaus R A. Elastases and elastin degradation. J Invest Dermatol 1982;79(Suppl 1):154s-159s.
Jackson S R, Avery N C, Tarlton J F, Eckford S D, Adrams P, Bailey A J. Changes in metabolism of collagen in genitourinary prolapse. The Lancet 1996; 347:1658-1661.
Lee B S and Nowak R A. Human leiomyoma smooth muscle cells show increased expression of transforming growth factor-beta 3 (TGF beta 3) and altered responses to the antiproliferative effects of TF beta. J Clin Endocrinol Metab. 2001; 86(2): 913-20
Moalli P A, Klingensmith W L, Meyn L A, Zyczynski H M. Regulation of matrix metalloproteinase expression by estrogen in fibroblasts that are derived from the pelvic floor. Am J Obstet Gynecol 2002;187:72-79.
Wiedow O, Schroder J M, Gregory H, Young J A, Christophers E. Elafin: An elastase-specific inhibitor of human skin. Purification, characterization, and complete amino acid sequence. J Biol Chem 1991;5:3356.
Helmstra, P S, Novel roles of protease inhibitors in infection and inflamrnation. Biochem Soc Trans 2002; 30 (part 2): 116-120.
Djurovic S, Os I, Hofstad A E, Abdelnoor M, Westheim A, Berg K. Increased plasma concentration of TGF-β1 after hormone replacement therapy. J Intern Med 2000;247:279-285
Loverro G, Perlino E, Maiorano E, Cormio G, Ricco R, Marra E, Nappi L, Gianni T, Selvaggi L. TGF-beta 1 and IGF-1 expression in atropic post-menopausal endometrium. Maturitas 1999;31:179-184
Yen S S C, Jaffe R B, eds., Reproductive Endocrinology 2^{nd} edition, 1986, pp. 213-214, WB Saunders Co., Philadelphia.
Bigazzi M, Nardi E. Prolactin and relaxin: Antagonism of the spontaneous motility of the uterus. J Clin Endocrinol Metab 1981;53:665.
Steinetz B G, O'Byrne E M, Kroc R L. The role of relaxin in cervical softening in mammals. In: Naftolin F, Stubblefield P G, eds. Dilation of the Uterine Cervix 1980, pp.157-177, Raven Press, New York.
Palejwala S, Stein D E, Weiss G, Monia B P, Tortoriello D, Goldsmith L T. Relaxin positively regulates matrix metalloproteinase expression in human lower uterine segment fibroblasts using a tyrosine kinase signaling pathway. Endocrinology 2001; 142(8): 3405-13.
Lenhart J A, Ryan P L, Ohleth K M, Palmer S S, Bagnell C A: Relaxin increases secretion of matrix metalloproteinase-2 and matrix metalloproteinase-9 during uterine and cervical growth and remodelling in the pig. Endocrinology 2001;142:3941-3949.
Unemori E N, Amento E P. Relaxin modulates synthesisi and secretion of procollagenase and collagen by human dermal fibroblasts. J Biol Chem 1990;265:10681-10685).
Mathrubutham M, Aybek Z, Fogarty J, Lee J, Rao S K, Badlani G H et al. Plasma elastase regulation in stress urinary incontinence. Neurourology and Urodynamics 1999;18:281-2.
Harvey M A, Johnston S L, Davies G A L. Second trimester serum relaxin concentrations are associated with pelvic organ prolapse following childbirth. J. of Pelvic Medicine & Surg. 2004;10(Suppl 1):S50.
Reddy G K and Enweneka, C S. A simplified method for the analysis of hydroxyproline in biological tissue. Clin. Biochem. 1996; 29(3): 225-9.
Maum Y and Morgan T E. A microassay for elastin. Annals Biochem. 1973; 53(2): 392-6.

1. A method of treatment of stress urinary incontinence (SUI) comprising administering an effective amount of an inhibitor of elastase activity to a pelvic supporting tissue of a patient in need of treatment.

2. The method of claim 1, wherein the inhibitor is selected from the group consisting of an endogenous inhibitor and a synthetic inhibitor.

3. The method of claim 2, wherein the inhibitor is an endogenous inhibitor selected from the group consisting of alpha-1 antitrypsin(ATT), elafin, secretory leukocyte protease inhibitor(SLPI), and alpha 2-macroglobulin.

4. The method of claim 2, wherein the inhibitor is selected from the group consisting of tissue inhibitors of metalloproteinases (TIMPs) and synthetic inhibitors of MMP-2 and MMP-9.

5. The method of claim 1, wherein the inhibitor is administered in a genetic construct comprising a polynucleotide squence coding for an endogenous elastase inhibitor.

6. The method of claim 5, wherein the inhibitor is ATT.

7. The method of claim 5, wherein the inhibitor is selected from the group consisting of elafin and SLPI.

8. The method of claim 1, wherein the inhibitor is administered by intravaginal or intrauterine routes.

9. The method of claim 1, wherein the inhibitor is administered to a patient at risk for developing SUI.

10. The method of claim 1, wherein the patient is afflicted with SUI.

11. A method of treatment of SUI comprising administering an effective amount of a relaxin antagonist to a pelvic supporting tissue of a patient in need of treatment.

12. The method of claim 11, wherein the antagonist inhibits the binding of relaxin to a relaxin receptor in the pelvic supporting tissue.

13. The method of claim 11, wherein the antagonist inhibits relaxin signalling in the pelvic supporting tissue.

14. The method of claim 11, wherein the antagonist is selected from the group consisting of peptides, antibodies and synthetic molecules.

15. The method of claim 11, wherein the antagonist is identified by screening in a cell-based assay.

16. The method of claim 11, wherein the treatment is prophylactic in a patient at risk for developing SUI.

17. A method for diagnosing SUI comprising incubating cultured pelvic fibroblasts from a test subject in the presence and absence of relaxin and comparing the level of ATT protein and total elastase activity in relaxin-treated and untreated cells, wherein a change in either one of the level of ATT or total elastase activity, but not both, is indicative of SUI, and wherein a unidirectional change in the level of both ATT and total elastase is indicative of normalcy.

18. The method of diagnosis according to claim 17, which further comprises comparing one or more of MMP-2, MMP-9 and TIMP-2 protein expression in the presence and absence of relaxin in test cells as compared with normal cells, wherein relaxin positively modulates MMP-2, MMP-9 and TIMP-2 protein expression in normal fibroblasts but does not modulate the expression of these proteins in fibroblasts from women with SUI.

19. The method of claim 17, wherein the cells are secretory phase cells.

20. The method of claim 17, which further comprises measuring mRNA levels of TIMP-1.
